(19) United States
(12) Patent Application Publication
(10) Pub. No.: US 2025/0268852 A1
(43) Pub. Date: Aug. 28, 2025

(54) MICROBIOTA IMPROVING AGENT

(71) Applicants: SANYO CHEMICAL INDUSTRIES, LTD., Kyoto (JP); JOYCLE CO., LTD., Kyoto (JP)

(72) Inventors: Ryosuke SUZUKI, Kyoto (JP); Naoya HIRAO, Kyoto (JP); Koji OTA, Kyoto (JP); Seiji HORIE, Kyoto (JP); Koji MIYAZAKI, Kyoto (JP)

(73) Assignees: SANYO CHEMICAL INDUSTRIES, LTD., Kyoto (JP); JOYCLE CO., LTD., Kyoto (JP)

(21) Appl. No.: 18/857,951

(22) PCT Filed: Apr. 20, 2023
(86) PCT No.: PCT/JP2023/015811

§ 371 Date: Oct. 18, 2024

(30) Foreign Application Priority Data
Apr. 21, 2022 (JP) 2022-070188
Dec. 27, 2022 (JP) 2022-210329

(51) Int. Cl. A61K 31/191; A61K 31/121; A61K 31/198; A61K 31/47; A61K 31/7012; A61K 33/14; A61K 33/24; A61K 33/42; A61K 38/05

(52) U.S. Cl. CPC A61K 31/191; A61K 31/121; A61K 31/198; A61K 31/47; A61K 31/7012; A61K 33/14; A61K 33/24; A61K 33/42; A61K 38/05

(57) ABSTRACT

The present invention aims to provide a microbiota improving agent for improving at least one of mucosal microbiota, skin microbiota, or nail microbiota, the microbiota improving agent exhibiting a high pathogenic bacteria inhibitory effect while maintaining resident non-pathogenic bacteria, thus persistently improving the microbiota balance. The present invention relates to a microbiota improving agent for improving at least one of mucosal microbiota, skin microbiota, or nail microbiota, the microbiota improving agent containing: a substance (X); and a base (Y) containing a non-volatile component, the substance (X) including at least one selected from the group consisting of a compound (X1) having two or more hydroxy groups contained in at least one oxoacid group and a salt of the compound (X1), a compound (X2) having one hydroxy group contained in an oxoacid group and a salt of the compound (X2), a heterocyclic compound (X3) having no oxoacid group and a salt of the compound (X3), and a ketone (X4) having no oxoacid group. In the compound (X1), a total mass percentage of the at least one oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 45% by mass or more based on a molecular weight of the compound (X1). The compound (X2) has an atom having an unshared electron pair, the atom excluding atoms contained in the oxoacid group and atoms contained in an amino group not constituting an imidazole skeleton and in an amino group not constituting a pyridine skeleton. In the compound (X2), a total mass percentage of the oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 55% by mass or more based on a molecular weight of the compound (X2). The heterocyclic compound (X3) has an imidazole skeleton or a pyridine skeleton and at least one of a hydroxy group or a carbonyl group, or has two or more skeletons selected from the group consisting of an imidazole skeleton and a pyridine skeleton. The ketone (X4) has a linear β-diketone structure. The substance (X) has a molecular weight of 550 or less. The non-volatile component has an HLB value of 7 or more. The microbiota improving agent satisfies the following conditions (i) and (ii): (i) the microbiota improving agent has a worked penetration of 100 to 400 as measured in accordance with JIS K 2220; and (ii) the microbiota improving agent has a viscosity at 37° C. of 1 to 4,000 Pa·s as measured with a cone-and-plate rheometer.

### TECHNICAL FIELD

The present invention relates to microbiota improving agents.

### BACKGROUND ART

Various bacteria coexist symbiotically in the human body (e.g., the mucosa (oral mucosa, etc.), skin, nails), and bacteria form a bacterial community called microbiota. The microbiota includes a mixture of so-called resident non-pathogenic bacteria and pathogenic bacteria.

The microbiota balance is usually maintained, which is known as a healthy state, and diseases caused by pathogenic bacteria are suppressed.

However, if the microbiota balance is disrupted due to factors such as diet, antibiotic intake, stress, aging, or poor hygiene, the numbers of pathogenic bacteria and opportunistic infectious bacteria increase, causing diseases. Therefore, in order to suppress diseases, it is important not only to inhibit pathogenic bacteria but also to maintain the healthy microbiota balance between pathogenic bacteria and resident non-pathogenic bacteria.

Known techniques relating to a pathogenic factor production inhibitor that exhibits a function of acting on specific harmful bacteria include a technique disclosed in Patent Literature 1.

### CITATION LIST

### Patent Literature

Patent Literature 1: JP 5716173 B

### SUMMARY OF INVENTION

### Technical Problem

However, as a result of investigation, it was found that the pathogenic factor production inhibitor described in Patent Literature 1 when used as is not only prevents or reduces the action of pathogenic bacteria, but also has a significant adverse effect on resident non-pathogenic bacteria. It is also difficult for the inhibitor to persistently prevent or reduce the action of pathogenic bacteria on the application site. Such a pathogenic factor production inhibitor is required to improve in order to persistently maintain the healthy microbiota balance.

Therefore, the present invention aims to provide a microbiota improving agent for improving at least one of mucosal microbiota, skin microbiota, or nail microbiota, the microbiota improving agent exhibiting a high pathogenic bacteria inhibitory effect while maintaining resident non-pathogenic bacteria, thus persistently improving the microbiota balance.

### Solution to Problem

The present inventors conducted extensive research to solve the above problems and arrived at the present invention. Specifically, the present invention relates to a microbiota improving agent for improving at least one of mucosal microbiota, skin microbiota, or nail microbiota, the microbiota improving agent containing:
a substance (X); and
a base (Y) containing a non-volatile component,
the substance (X) including at least one selected from the group consisting of
a compound (X1) having two or more hydroxy groups contained in at least one oxoacid group and a salt of the compound (X1),
a compound (X2) having one hydroxy group contained in an oxoacid group and a salt of the compound (X2),
a heterocyclic compound (X3) having no oxoacid group and a salt of the compound (X3), and
a ketone (X4) having no oxoacid group,
wherein in the compound (X1), a total mass percentage of the at least one oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 45% by mass or more based on a molecular weight of the compound (X1),
the compound (X2) has an atom having an unshared electron pair, the atom excluding atoms contained in the oxoacid group and atoms contained in an amino group not constituting an imidazole skeleton and in an amino group not constituting a pyridine skeleton,
in the compound (X2), a total mass percentage of the oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 55% by mass or more based on a molecular weight of the compound (X2),
the heterocyclic compound (X3) has an imidazole skeleton or a pyridine skeleton and at least one of a hydroxy group or a carbonyl group, or has two or more skeletons selected from the group consisting of an imidazole skeleton and a pyridine skeleton,
the ketone (X4) has a linear β-diketone structure,
the substance (X) has a molecular weight of 550 or less,
the non-volatile component has an HLB value of 7 or more, and
the microbiota improving agent satisfies the following conditions (i) and (ii):
(i) the microbiota improving agent has a worked penetration of 100 to 400 as measured in accordance with JIS K 2220; and
(ii) the microbiota improving agent has a viscosity at 37° C. of 1 to 4,000 Pa·s as measured with a cone-and-plate rheometer.

### Advantageous Effects of Invention

The present invention provides a microbiota improving agent for improving at least one of mucosal microbiota, skin microbiota, or nail microbiota, the microbiota improving agent exhibiting a high pathogenic bacteria inhibitory effect while maintaining resident non-pathogenic bacteria, thus persistently improving the microbiota balance.

### DESCRIPTION OF EMBODIMENTS

The microbiota improving agent of the present invention for improving at least one of mucosal microbiota, skin microbiota, or nail microbiota (hereinafter also referred to as “microbiota improving agent of the present invention”) contains a substance (X) and a base (Y) containing a non-volatile component, the substance (X) including at least one selected from the group consisting of a compound (X1) having two or more hydroxy groups contained in at least one oxoacid group and a salt of the compound (X1), a compound (X2) having one hydroxy group contained in an oxoacid group and a salt of the compound (X2), a heterocyclic compound (X3) having no oxoacid group and a salt of the compound (X3), and a ketone (X4) having no oxoacid group.

The microbiota improving agent of the present invention exhibits a high pathogenic bacteria inhibitory effect while maintaining resident non-pathogenic bacteria, thus improving the microbiota balance.

Here, the “high pathogenic bacteria inhibitory effect” means at least one of killing harmful bacteria, inactivating harmful bacteria and inhibiting their growth, or inhibiting harmful bacteria from producing toxic substances (pathogenic factors).

The term “pathogenic bacteria” herein refers to bacteria, fungi, and the like that are highly pathogenic and cause various infectious diseases and the like as they grow. Specific examples thereof include Staphylococcus aureus, Streptococcus pyogenes, Pseudomonas aeruginosa, Porphyromonas gingivalis, Fusobacterium nucleatum, Prevotella intermedia, Trichophyton rubrum, Trichophyton mentagrophytes, Candida albicans, and Candida glabrata.

The term “resident non-pathogenic bacteria” refers to resident bacteria that inhibit the growth of pathogenic bacteria and maintain health. Specific examples thereof include Staphylococcus epidermidis and Streptococcus salivarius.

Of the above-listed pathogenic bacteria, Porphyromonas gingivalis, Fusobacterium nucleatum, and Prevotella intermedia are known as pathogenic bacteria that can occur in the oral cavity.

Of the above-listed pathogenic bacteria, Trichophyton rubrum, Trichophyton mentagrophytes, Staphylococcus aureus, Candida albicans, and Candida glabrata are known as pathogenic bacteria that can occur in the skin and nails.

Of the above-listed non-pathogenic bacteria, Streptococcus salivarius is known as a resident bacteria present in the oral cavity.

Of the above-listed non-pathogenic bacteria, Staphylococcus epidermidis is known as a resident bacteria present in the skin and nails.

The microbiota improving agent of the present invention can selectively inhibit the quorum sensing mechanism (i.e., the mechanism that produces autoinducers) of pathogenic bacteria, and has a high pathogenic bacteria inhibitory effect while maintaining resident non-pathogenic bacteria.

### Substance (X)

The microbiota improving agent of the present invention contains a substance (X).

The substance (X) includes at least one selected from the group consisting of a compound (X1) having two or more hydroxy groups contained in at least one oxoacid group and a salt of the compound (X1), a compound (X2) having one hydroxy group contained in an oxoacid group and a salt of the compound (X2), a heterocyclic compound (X3) having no oxoacid group and a salt of the compound (X3), and a ketone (X4) having no oxoacid group.

The substance (X) may include two or more of the compounds (X1), two or more of the compounds (X2), two or more of the heterocyclic compounds (X3), or two or more of the ketones (X4). The substance (X) may include two or more salts of the compound(s) (X1), two or more salts of the compound(s) (X2), or two or more salts of the compound (s) (X3).

The compound (X1) has two or more hydroxy groups contained in at least one oxoacid group. The compound (X1) may have two or more oxoacid groups.

Herein, the term “oxoacid group” refers to a group having a structure in which a hydroxy group (—OH) and an oxo group (═O) are bonded to a single central atom (such as a carbon atom, a phosphorus atom, a sulfur atom, or a tungsten atom). One oxoacid group may contain two or more hydroxy groups or two or more oxo groups.

For example, triphosphate, which is represented by the following formula:

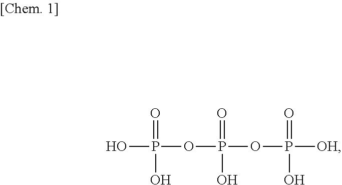 [SMILES: O=P(O)(O)OP(=O)(O)OP(=O)(O)O]

contains two different oxoacid groups, i.e., oxoacid groups each represented by —P(═O)(OH)₂ at both ends of the molecule and an oxoacid group represented by —P(═O)(OH)— in the center of the molecule. The oxoacid groups at both ends each contain two hydroxy groups.

From the viewpoint of the effect of the microbiota improving agent, in the compound (X1), the total mass percentage of the at least one oxoacid group, a hydroxy group not contained in the oxoacid group, a carbonyl group (C═O) not contained in the oxoacid group, carbon atoms contained in an imidazole skeleton and a pyridine skeleton, and nitrogen atoms contained in the imidazole skeleton and the pyridine skeleton is 45% by mass or more, preferably 55% by mass or more, based on the molecular weight of the compound (X1). When the compound (X1) in the microbiota improving agent includes multiple compounds, each compound satisfies this condition. Herein, the mass of an oxoacid group is the total mass of the central atom, the oxo group(s), and the hydroxy group(s).

For example, malic acid, which has a molecular weight of 134.1 and is represented by the following formula:

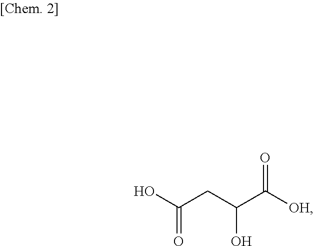

has two carboxy groups (formula weight 45.0), which are oxoacid groups, and one hydroxy group (formula weight 17.0) not contained in the oxoacid groups. The total mass percentage of these groups based on the molecular weight of malic acid is calculated as follows: [(45.0×2+17.0×1)/134.1]×100=80 (% by mass).

The compound (X1) has at least one oxoacid group as an essential group, and optionally has a hydroxy group not contained in the oxoacid group, a carbonyl group not contained in the oxoacid group, an imidazole skeleton, and a pyridine skeleton as non-essential components.

Herein, the “imidazole skeleton” refers to a structure represented by the following formula:

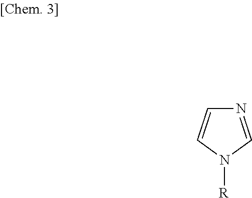

wherein R is a hydrogen atom or a group capable of bonding to imidazole.

The “pyridine skeleton” refers to a structure represented by the following formula:

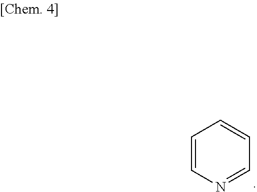

These structures may be contained in a fused ring.

From the viewpoint of a further improvement in the effect of the microbiota improving agent, in the compound (X1), the total mass percentage of the at least one oxoacid group, a hydroxy group not contained in the oxoacid group, and a carbonyl group not contained in the oxoacid group is 45% by mass or more based on the molecular weight of the compound (X1).

Examples of the compound (X1) include polyphosphoric acid, tungstic acid (H₂WO₄), ethylenediaminetetraacetic acid, glutamic acid, iminodiacetic acid, malic acid, citric acid, tartaric acid, adenosine triphosphate, and guanosine triphosphate. Herein, tungstic acid is considered to be a compound having two hydroxy groups contained in an oxoacid group, as shown in the following chemical formula (1), and is thus included in the compound (X1).

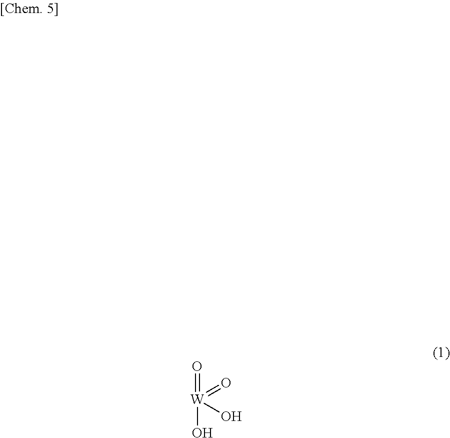

From the viewpoint of the effect of the microbiota improving agent, preferably, the compound (X1) has no “—NH₂” group.

When the compound (X1) is a hydroxycarboxylic acid, the ratio of the number of moles of hydroxy groups to the number of moles of carboxy groups (—COOH) (number of moles of —OH/number of moles of —COOH) in the hydroxycarboxylic acid is preferably ⅓ or more, more preferably ½ or more, from the viewpoint of the effect of the microbiota improving agent.

Non-limiting examples of the salt of the compound (X1) include inorganic acid salts such as hydrochlorides, sulfates, nitrates, and phosphates; organic acid salts such as acetates, citrates, maleates, malates, oxalates, lactates, succinates, fumarates, and propionates; alkali metal salts such as sodium salts and potassium salts; alkaline earth metal salts such as calcium salts and magnesium salts; ammonium salts; salts with organic bases such as triethylamine, triethanolamine, and pyridine; and amino acid salts with basic amino acids (e.g., arginine) or with acidic amino acids (e.g., glutamic acid). Of these, sodium salts and potassium salts are preferred.

The compound (X2) has an atom having an unshared electron pair, the atom excluding the atoms contained in the oxoacid group and the atoms contained in an amino group not constituting an imidazole skeleton and in an amino group not constituting a pyridine skeleton.

Preferred examples of the atom having an unshared electron pair include the oxygen atom contained in a carbonyl group, the oxygen atom contained in a hydroxy group (excluding the oxygen atoms contained in an oxoacid group), the nitrogen atom contained in an imidazole skeleton, and the nitrogen atom contained in a pyridine skeleton.

From the viewpoint of the effect of the microbiota improving agent, in the compound (X2), the total mass percentage of the oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 55% by mass or more based on the molecular weight of the compound (X2). When the compound (X2) in the microbiota improving agent includes multiple compounds, each compound satisfies this condition.

The compound (X2) has an oxoacid group as an essential group, and optionally has a hydroxy group not contained in the oxoacid group, a carbonyl group not contained in the oxoacid group, an imidazole skeleton, and a pyridine skeleton.

Examples of the compound (X2) include a compound (X21) having no amino group and a compound (X22) having an amino group.

Examples of the compound (X21) having no amino group include hydroxycarboxylic acids (gluconic acid, glucuronic acid, etc.) and keto acids (levulinic acid, etc.).

Examples of the compound (X22) having an amino group include glycylglycine and histidine.

Examples of the salt of the compound (X2) include those described as the salt of the compound (X1), and the same applies to preferred examples thereof.

The heterocyclic compound (X3) has an imidazole skeleton or a pyridine skeleton and at least one of a hydroxy group or a carbonyl group, or has two or more skeletons selected from the group consisting of an imidazole skeleton and a pyridine skeleton.

Examples of the heterocyclic compound (X3) include 8-quinolinol (a compound having one pyridine skeleton and one hydroxy group) and phenanthroline (a compound having two pyridine skeletons).

From the viewpoint of the effect of the microbiota improving agent, in the heterocyclic compound (X3), the total mass percentage of the carbon atoms contained in the imidazole skeleton and the pyridine skeleton, the nitrogen atoms contained in the imidazole skeleton and the pyridine skeleton, the hydroxy group(s), and the carbonyl group(s) is preferably 50% by mass or more based on the molecular weight of the heterocyclic compound (X3). When the heterocyclic compound (X3) in the microbiota improving agent includes multiple compounds, each compound preferably satisfies this condition.

Examples of the salt of the heterocyclic compound (X3) include those described as the salt of the compound (X1).

The ketone (X4) has a linear β-diketone structure.

The ketone (X4) may be a salt having a counter ion.

Examples of the ketone (X4) include benzoylacetone and acetylacetone.

From the viewpoint of the effect of the microbiota improving agent, in the ketone (X4), the total mass percentage of the carbonyl groups is preferably 30% by mass or more based on the molecular weight of the ketone (X4) (in the case where the ketone (X4) is a salt having a counter ion, it is based on the mass of only an ion having a linear β-diketone structure excluding the counter ion). When the ketone (X4) in the microbiota improving agent includes multiple compounds, each compound preferably satisfies this condition.

From the viewpoint of safety, the substance (X) preferably includes at least one selected from the group consisting of levulinic acid and salts thereof, tungstic acid and salts thereof, benzoylacetone, polyphosphoric acid and salts thereof, and acetylacetone. Polyphosphoric acid (the number of repeating phosphate units is preferably 2 to 5, more preferably 3 to 5) and salts thereof are more preferred, and pentasodium triphosphate is particularly preferred.

From the viewpoint of the effect of the microbiota improving agent, the substance (X) has a molecular weight of 550 or less. When the substance (X) in the microbiota improving agent includes multiple compounds, each compound has a molecular weight of 550 or less.

As for the substance (X), the amount of magnesium captured per 100 g of the substance (X) is preferably 1 g or more, as measured by the ion-selective electrode method.

When the substance (X) is a hydrate, 100 g of the substance (X) refers to 100 g by weight excluding hydration water.

The measurement by the ion-selective electrode method is performed using 2 mM of the substance (X) relative to a 1 mM aqueous MgCl₂ solution having a temperature of 20° C. and a pH of 7.

### Base (Y)

The microbiota improving agent of the present invention contains not only the substance (X) but also a base (Y) containing a non-volatile component. The substance (X) does not correspond to a component contained in the base (Y).

The non-volatile component in the base (Y) has an HLB value of 7 or more. If the HLB value of the non-volatile component is less than 7, the spreadability of the microbiota improving agent when applied to the affected area is poor. The HLB value of the non-volatile component is preferably 7 to 60 from the viewpoint of spreadability. A component having an HLB value of 7 or more in the microbiota improving agent is regarded as the non-volatile component in the base (Y).

The HLB value is an index showing the balance between hydrophilicity and lipophilicity. The HLB value is known as a value calculated by the Oda method described in, for example, Takehiko Fujimoto, “Introduction to Surfactants (Kaimen Kakuseizai Nyumon)”, Sanyo Chemical Industries, Ltd., 2007, p. 212, and is not a value calculated by the Griffin method.

The HLB value of a compound as a non-volatile component can be calculated based on the ratio between the organic value and the inorganic value of the compound by the following formula:

HLB=10×inorganic value/organic value.

Regarding the organic values and the inorganic values for giving HLB values, the organic values are set to 20 per carbon atom, and the inorganic values are calculated using the values shown in the table on page 213 of the above-described “Introduction to Surfactants (Kaimen Kakuseizai Nyumon)” (“Value” of Inorganic group or “Value” of “Inorganic value” of Organic and inorganic group). Examples of the calculation include:
—CH₃ group: organic value 20, inorganic value 0;
—CH₂— group: organic value 20, inorganic value 0;
═CH₂ group: organic value 20, inorganic value 1;
═CH— group: organic value 10, inorganic value 1;
benzene ring: organic value 120, inorganic value 15;
—O— group: inorganic value 20;
—COO—: organic value 20, inorganic value 60;
—OH group: inorganic value 100; and
—COOH group: organic value 20, inorganic value 150.

In the present invention, the HLB values are calculated using the following organic values and inorganic values for the following components.
COO⁻M⁺: organic value 20, inorganic value 400 (“M⁺” is a counter ion of “COO⁻” and represents a metal cation or ammonium cation)
Si atom: organic value 0, inorganic value 0

When the non-volatile component includes multiple components, the HLB value of the non-volatile component is determined by the weighted average of the HLB values of the components based on the weight percentages of the components.

The “non-volatile component” in the present invention refers to the residue remaining after a sample is heated and dried in an uncovered glass petri dish at 130° C. for 45 minutes in a circulating air dryer.

From the viewpoint of the effect of the microbiota improving agent, the amount (weight percentage) of the non-volatile component is preferably 0.1 to 80% by weight, more preferably 1 to 70% by weight, based on the weight of the microbiota improving agent.

Furthermore, from the viewpoint of the effect of the microbiota improving agent, the amount (weight percentage) of the non-volatile component is preferably 30 to 6,000% by weight, more preferably 50 to 5,000% by weight, relative to the weight of the substance (X).

The non-volatile component in the base (Y) is preferably a polymer having a weight average molecular weight of 10,000 to 3,000,000. Furthermore, the polymer having a weight average molecular weight of 10,000 to 3,000,000 preferably contains a compound having a molecular weight of 500 or more. A polymer contained in the microbiota improving agent and having a weight-average molecular weight of 10,000 to 3,000,000 and an HLB value of 7 or more is regarded as the non-volatile component in the base (Y).

The weight-average molecular weight herein can be measured by gel permeation chromatography (GPC) under the following conditions.

### <GPC Measurement Conditions>

(1) Apparatus: gel permeation chromatography [model number “HLC-8120GPC” available from Tosoh Corporation]
(2) Column: “TSKgel G6000PWxl” and “TSKgel G3000PWxl” connected in series, both available from Tosoh Corporation
(3) Eluent: a solution prepared by dissolving 0.5% by weight sodium acetate in methanol/water=30/70 (volume ratio)
(4) Reference substance: polyethylene glycol (hereinafter abbreviated as PEG)
(5) Injection conditions: sample concentration of 0.25% by weight, column temperature of 40° C.

Examples of monomers to form the polymer include monosaccharides such as α-glucose, β-glucose, and uronic acids (e.g., mannuronic acid, guluronic acid); (meth)acrylic monomers such as C3-C10 (meth)acrylic monomers (e.g., acrylic acid); (alkyl)alkoxysilanes such as C4-C40 dialkyldialkoxysilanes (e.g., diethoxydimethylsilane, dimethoxydimethylsilane); and alkylene oxides such as C2-C4 alkylene oxides (e.g., ethylene oxide, propylene oxide, butylene oxide).

In the present invention, (meth)acrylic refers to acrylic and/or methacrylic.

In the present invention, (alkyl)alkoxysilanes refer to alkoxysilanes and/or alkylalkoxysilanes.

Specific examples of the polymer include carboxyalkylcellulose and salts thereof (e.g., carboxymethylethylcellulose, sodium carboxymethylcellulose), alkylcellulose and salts thereof (e.g., methylcellulose 4000), sodium alginate, xanthan gum, starch-sodium acrylate graft copolymers, polyalkylene glycols such as polyethylene glycols (e.g., PEG 20000), polydimethylsiloxane (A), and an adduct of alkylene oxide with aliphatic alcohol (B).

The non-volatile component may consist of one of the polymers or may include two or more thereof in combination.

For example, the non-volatile component may include the polydimethylsiloxane (A) and the adduct of alkylene oxide with aliphatic alcohol (B) in combination. In this case, the weighted average of the HLB values of the polydimethylsiloxane (A) and the adduct of alkylene oxide with aliphatic alcohol (B) [weighted average based on the weight percentages of polydimethylsiloxane (A) and the adduct of alkylene oxide with aliphatic alcohol (B)] is 7 or more.

The base (Y) may contain only a non-volatile component, or may contain a volatile component in addition to a non-volatile component.

The following describes as an example of the base (Y) a composition containing the polydimethylsiloxane (A) and the adduct of alkylene oxide with aliphatic alcohol (B) as non-volatile components and water as a volatile component (an additive described later).

Examples of the polydimethylsiloxane (A) include those having a structure in which a dimethylsiloxane unit {—(CH₃)₂SiO-} is a repeating unit. Specific examples thereof include those represented by the following formula (1):

CH₃—[(CH₃)₂SiO]ₙ—Si—(CH₃)₃ (1)

wherein n is the number-average number of dimethylsiloxane units and is a number from 60 to 500, but is not necessarily an integer.

In the formula (1), n is preferably a number from 100 to 450, more preferably a number from 150 to 400, from the viewpoint of spreadability.

Examples of commercially available products of the polydimethylsiloxane (A) include Dow Corning® Q7-9120 Silicone Fluid, 100 cSt (n=85), 350 cSt (n=200), and 1000 cSt (n=350), available from Dow Corning Toray Co., Ltd.

From the viewpoint of the persistence of the effect of the microbiota improving agent, the polydimethylsiloxane (A) is preferably polydimethylsiloxane represented by the formula (1).

From the viewpoint of spreadability, the adduct of alkylene oxide with aliphatic alcohol (B) is preferably one represented by the following formula (2):

R¹—(OA)ₘ-OH (2)

wherein R¹ represents a C8-C18 alkyl group or a C8-C18 alkenyl group; OA represents a C2-C4 oxyalkylene group; and m represents the average number of moles of oxyalkylene groups added and is a number from 1 to 50, but is not necessarily an integer.

In the formula (2), R¹ represents a C8-C18 alkyl group or a C8-C18 alkenyl group.

The C8-C18 alkyl group may be a linear C8-C18 alkyl group or a branched C8-C18 alkyl group.

Examples of the linear C8-C18 alkyl group include n-octyl, n-decyl, n-undecyl, n-dodecyl, n-tridecyl, n-tetradecyl, n-pentadecyl, n-hexadecyl, n-heptadecyl, and n-octadecyl groups.

Examples of the branched C8-C18 alkyl group include 2-ethylhexyl, isodecyl, isoundecyl, isododecyl, isotridecyl, isotetradecyl, isopentadecyl, isohexadecyl, isoheptadecyl, and isooctadecyl groups.

The C8-C18 alkenyl group may be a linear C8-C18 alkenyl group or a branched C8-C18 alkenyl group.

Examples of the linear C8-C18 alkenyl group include n-octenyl, n-dodecenyl, n-hexadecenyl, and n-octadecenyl groups.

Examples of the branched C8-C18 alkenyl group include isodecenyl, isododecenyl, and isooctadecenyl groups.

From the viewpoint of spreadability, R¹ is preferably a linear C8-C18 alkyl group, more preferably a linear C10-C18 alkyl group.

Examples of OA in the formula (2) include oxyethylene, oxypropylene, and oxybutylene groups.

From the viewpoint of spreadability, the OA is preferably an oxyethylene group.

In the formula (2), m is preferably a number from 3 to 40 from the viewpoint of spreadability.

From the viewpoint of spreadability, the HLB value of the adduct of alkylene oxide with aliphatic alcohol (B) is preferably 8.5 to 11.0, more preferably 9.0 to 10.5.

From the viewpoints of spreadability and persistence of the effect, the weight ratio of polydimethylsiloxane (A) to adduct of alkylene oxide with aliphatic alcohol (B) {(A)/(B)} is preferably 85/15 to 40/60, more preferably 80/20 to 35/65.

From the viewpoint of the persistence of the effect, the total amount of the polydimethylsiloxane (A) and the adduct of alkylene oxide with aliphatic alcohol (B) is preferably 0.1 to 85% by weight, more preferably 1 to 85% by weight, still more preferably 5 to 85% by weight, particularly preferably 40 to 85% by weight, most preferably 50 to 80% by weight, based on the weight of the microbiota improving agent.

From the viewpoint of the persistence of the effect, the total amount of the polydimethylsiloxane (A) and the adduct of alkylene oxide with aliphatic alcohol (B) is preferably 30 to 99% by weight, more preferably 50 to 99% by weight, particularly preferably 80 to 99% by weight, most preferably 90 to 99% by weight, based on the total weight of the substance (X) and the non-volatile component in the base (Y).

When the microbiota improving agent contains only the four components of the substance (X), the polydimethylsiloxane (A), the adduct of alkylene oxide with aliphatic alcohol (B), and water, with the three components other than the substance (X) serving as the base (Y), the total amount of the polydimethylsiloxane (A) and the adduct of alkylene oxide with aliphatic alcohol (B) is preferably 1 to 85% by weight, more preferably 5 to 85% by weight, particularly preferably 40 to 85% by weight, most preferably 50 to 80% by weight, based on the weight of the base (Y), from the viewpoint of the persistence of the effect.

An example of a method for producing the base (Y) containing the polydimethylsiloxane (A), the adduct of alkylene oxide with aliphatic alcohol (B), and water is a method including a step (I) of mixing the polydimethylsiloxane (A), the adduct of alkylene oxide with aliphatic alcohol (B), and water.

Preferably, the polydimethylsiloxane (A) and the adduct of alkylene oxide with aliphatic alcohol (B) are mixed in advance. The mixing of the polydimethylsiloxane (A) and the adduct of alkylene oxide with aliphatic alcohol (B) is preferably performed at a temperature of 20° C. to 70° C., more preferably 25° C. to 65° C., from the viewpoint of emulsifiability.

In the step (I), the weight ratio of polydimethylsiloxane (A) to adduct of alkylene oxide with aliphatic alcohol (B) {(A)/(B)} is preferably 85/15 to 40/60, more preferably 80/20 to 35/65, from the viewpoints of spreadability and persistence of the effect.

In the step (I), the temperature of the water is preferably 20° C. to 70° C., more preferably 25° C. to 65° C., from the viewpoint of emulsifiability.

In the step (I), the amount of water is preferably 15 to 99% by weight, more preferably 20 to 99% by weight, based on the total weight of the polydimethylsiloxane (A), the adduct of alkylene oxide with aliphatic alcohol (B), and water, from the viewpoint of the persistence of the effect.

In the step (I), the mixing is preferably performed by stirring with a planetary mixer, from the viewpoint of gelation.

From the viewpoint of uniformity, the rotation speed of the planetary mixer is preferably 10 to 70 rpm for rotation and 5 to 40 rpm for revolution, more preferably 15 to 60 rpm for rotation and 10 to 35 rpm for revolution.

Examples of the stirring blade include a screw blade and a gate blade. From the viewpoint of uniformity, a gate blade is preferred.

In the step (I), when a mixture of the polydimethylsiloxane (A) and the adduct of alkylene oxide with aliphatic alcohol (B) is mixed with water, the water is preferably added in 5 to 50 portions, more preferably in 10 to 40 portions, from the viewpoint of gelation.

Also, in the step (I), from the viewpoint of gelation, water is preferably mixed as follows: a portion of water is added, and the appearance is confirmed to be sufficiently uniform, followed by adding another portion of water.

When the microbiota improving agent of the present invention is used for improving the oral microbiota balance, from the viewpoint of the persistence of the effect, the base (Y) preferably contains (Y-A) and/or (Y-B) as a non-volatile component, which are described later, and more preferably contains both the (Y-A) and the (Y-B).

### <(Y-A)>

The (Y-A) is a polymer having an anionic group such as a carboxy group or a carboxylate group (e.g., sodium carboxymethylcellulose, xanthan gum, sodium alginate) and having a weight average molecular weight of 50,000 to 3,000,000, preferably 100,000 to 2,000,000. The (Y-A) may be a mixture of two or more of such polymers.

### <(Y-B)>

The (Y-B) is a component having an HLB value of 7 or more excluding the (Y-A) and satisfies the following conditions (1) and/or (2):
(1) a solution having a concentration of 1% by weight of the (Y-B) prepared by dilution with water has a viscosity of 1 mPa·s or higher and lower than 500 mPa·s, preferably 5 mPa·s or higher and 450 mPa·s or lower (the viscosity is measured in the same manner as the viscosity at 37° C. of the microbiota improving agent measured with a cone-and-plate rheometer, which is described later); and
(2) the weight average molecular weight is 10,000 to 80,000, preferably 15,000 to 50,000.

The (Y-B) may be a mixture of two or more of the above components.

When the (Y-A) and (Y-B) are contained, the ratio of the weight of the (Y-A) to the weight of the (Y-B) [(Y-A)/(Y-B)] is preferably 0.001 to 0.2, more preferably 0.001 to 0.1.

### Other Components

The microbiota improving agent of the present invention may contain a component (additive) other than and in addition to the substance (X) and non-volatile components. The additive may be added separately from the base (Y) or may be appropriately contained in the base (Y).

Examples of the additive include lubricants such as wax, antioxidants, ultraviolet absorbers, antifoaming agents, preservatives, fragrances, and solvents such as water and organic solvents (e.g., ethanol).

When the additive contains the following bactericidal component, the total weight of the bactericidal component is preferably 2% by weight or less relative to the total weight of the substance (X), from the viewpoint of the effect of the microbiota improving agent.

The bactericidal component includes at least one selected from the group consisting of glycyrrhizinic acid and salts thereof, β-glycyrrhetinic acid, isopropylmethylphenol, cetylpyridinium chloride, benzalkonium chloride, benzethonium chloride, alkyldiaminoethylglycine chloride solution, chlorhexidine hydrochloride, chlorhexidine hydrochloride, triclosan, lysozyme chloride, hinokitiol, sodium lauroyl sarcosinate, and sodium lauryl sulfate.

The amount of the solvent in the microbiota improving agent is not limited as long as it allows the viscosity and the worked penetration of the microbiota improving agent to fall within the ranges described below. The amount is preferably, for example, 5 to 99% by weight based on the total weight of the microbiota improving agent.

The amount of water in the microbiota improving agent is preferably 5 to 99% by weight, more preferably 30 to 99% by weight, based on the total weight of the microbiota improving agent, from the viewpoints of spreadability and a microbiota balance improving effect.

In the case of the microbiota improving agent containing water, the microbiota improving agent produced by the method described below and allowed to stand at 25° C. for 10 minutes preferably has a visually uniform appearance (the agent is not separated into two or more phases).

The upper limit of the amount of additives other than the solvent in the microbiota improving agent is preferably 10% by weight or less, more preferably 8% by weight or less, particularly preferably 1% by weight or less, based on the weight of non-volatile component(s) in the microbiota improving agent.

The lower limit of the amount of additives other than the solvent in the microbiota improving agent is, for example, 0.1% by weight or more based on the weight of non-volatile component(s) in the microbiota improving agent, but is not limited thereto.

### Microbiota Improving Agent

The amount (weight percentage) of the substance (X) is preferably 0.01 to 5% by weight, more preferably 0.05 to 3% by weight, based on the weight of the microbiota improving agent.

From the viewpoint of suitably imparting a high pathogenic bacteria inhibitory effect, the lower limit of the amount (weight percentage) of the substance (X) is preferably 0.01% by weight, more preferably 0.05% by weight, based on the weight of the microbiota improving agent of the present invention.

From the viewpoint of suitably maintaining resident non-pathogenic bacteria, the upper limit of the amount of the substance (X) is preferably 5% by weight, more preferably 3% by weight, based on the weight of the microbiota improving agent of the present invention.

When a hydroxycarboxylic acid (a compound having a hydroxy group and a carboxy group, particularly citric acid) is used as the compound (X2), the weight percentage of the hydroxycarboxylic acid as the compound (X2) is preferably 1% by weight or more based on the weight of the microbiota improving agent, from the viewpoint of the effect of the microbiota improving agent.

The lower limit of the amount of the substance (X) is preferably 1% by weight, more preferably 2% by weight, relative to the weight of non-volatile component(s) in the microbiota improving agent. The upper limit of the amount of the substance (X) is preferably 200% by weight, more preferably 150% by weight, relative to the weight of non-volatile component(s) in the microbiota improving agent. The amount of the substance (X) is preferably 1 to 200% by weight, more preferably 2 to 150% by weight, relative to the weight of non-volatile component(s) in the microbiota improving agent.

The microbiota improving agent containing the substance (X) in an amount within the above ranges has a high pathogenic bacteria inhibitory effect while maintaining resident non-pathogenic bacteria, and can suitably impart the effect of maintaining the above-described performances for a long period of time.

In the case where the microbiota improving agent of the present invention contains zinc atoms and/or magnesium atoms (including the case where the agent contains a compound containing any of these atoms such as zinc oxide), the total weight percentage of zinc atoms and magnesium atoms is preferably 0.5% by weight or less relative to the total weight of the substance (X), from the viewpoint of the effect of the microbiota improving agent.

In the case where the microbiota improving agent of the present invention contains zinc atoms and/or magnesium atoms (including the case where the agent contains a compound containing any of these atoms), the total weight percentage of zinc atoms and magnesium atoms is preferably 0.005% by weight or less based on the weight of the microbiota improving agent, from the viewpoint of the effect of the microbiota improving agent.

The microbiota improving agent of the present invention has a worked penetration of 100 to 400 as measured in accordance with JIS K 2220. If the worked penetration is less than 100, the pathogenic bacteria inhibitory effect is poor. If the worked penetration exceeds 400, resident non-pathogenic bacteria are not maintained easily.

The worked penetration of the microbiota improving agent of the present invention, measured in accordance with JIS K 2220, is preferably 100 to 300, from the viewpoint of the persistence of the effect.

The viscosity at 37° C. of the microbiota improving agent of the present invention, measured with a cone-and-plate rheometer, is 1 to 4,000 Pa·s.

Specifically, the viscosity is measured by the following measurement method.

### <Measurement Conditions>

Device: MCR302 (Anton Paar)
Jig: CP-25 (cone-and-plate diameter: 25 mm)
Gap: 1 mm
Temperature: 37° C.
Strain rate: 0.5 (1/s) (rotation speed: 0.6 rpm)

### <Measurement Method>

Approximately 1.5 g of a sample is placed on the sample stage and sandwiched between the cone and the plate of CP-25, with the gap therebetween being set to 1 mm. The excess sample from the cone and plate is scraped off.

The temperature is adjusted to and maintained at 37° C. for one minute, and measurements are performed while changing the rotation speed from 0.1 to 10 rpm (20 plots at measurement intervals of 10 seconds to 1 second). The viscosity value when the strain rate is 0.5 (1/s) (rotation speed: 0.6 rpm) is recorded and taken as the viscosity of the microbiota improving agent.

If the viscosity of the microbiota improving agent is lower than 1 Pa·s, the persistence of the effect of the microbiota improving agent is poor. If the viscosity of the microbiota improving agent is higher than 4,000 Pa·s, the pathogenic bacteria inhibitory effect is poor.

From the viewpoint of the persistence of the effect, the viscosity of the microbiota improving agent is preferably 10 to 4,000 Pa·s, more preferably 1,000 to 4,000 Pa·s.

From the viewpoint of spreadability, the viscosity of the microbiota improving agent is preferably 2,000 to 3,700 Pa·s.

A solution having a concentration of 1% by weight of the microbiota improving agent of the present invention prepared by diluting the microbiota improving agent with water (e.g., ion-exchanged water) preferably has a pH of 6 to 8, from the viewpoint of the microbiota balance improving effect.

The pH can be measured using a pH meter at 25° C.

The microbiota improving agent of the present invention can be produced by adding the components and uniformly mixing them at room temperature or, if necessary, while heating (e.g., 30° C. to 70° C.). The components may be added in any order and by any method.

### Method of Use

The microbiota improving agent of the present invention is a microbiota improving agent for improving at least one of mucosal microbiota, skin microbiota, or nail microbiota. The microbiota improving agent can be used as follows: it is applied to the affected area [e.g., mucosa such as oral cavity mucosa (tongue, gums, gingiva, etc.), intestinal mucosa, or eye mucosa, skin, nails (nail plates)] or the vicinity of the affected area with a finger or the like and is left at the affected area. In particular, it is preferably applied to mucosa.

For example, when the microbiota improving agent of the present invention is applied to the oral cavity mucosa (tongue, gums, gingiva, etc.), it is expected to be effective in preventing periodontal disease, which progresses through the stages of gingivitis and periodontitis.

Use of non-volatile components appropriate to the application allows the microbiota improving agent of the present invention to be used as pharmaceuticals, medical devices (medical adhesives, wound healing materials, etc.), quasi-drugs, cosmetics, foods, etc. Use of the agent for these applications can achieve improvement of the microbiota balance in the applied area. Specific examples of the applications include toothpastes, oral gels, oral care foods, supplements, pet care products, and nail care products.

The present specification discloses the following.

The present disclosure (1) relates to a microbiota improving agent for improving at least one of mucosal microbiota, skin microbiota, or nail microbiota, the microbiota improving agent containing:
a substance (X); and
a base (Y) containing a non-volatile component,
the substance (X) including at least one selected from the group consisting of
a compound (X1) having two or more hydroxy groups contained in at least one oxoacid group and a salt of the compound (X1),
a compound (X2) having one hydroxy group contained in an oxoacid group and a salt of the compound (X2),
a heterocyclic compound (X3) having no oxoacid group and a salt of the compound (X3), and
a ketone (X4) having no oxoacid group,
wherein in the compound (X1), a total mass percentage of the at least one oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 45% by mass or more based on a molecular weight of the compound (X1),
the compound (X2) has an atom having an unshared electron pair, the atom excluding atoms contained in the oxoacid group and atoms contained in an amino group not constituting an imidazole skeleton and in an amino group not constituting a pyridine skeleton,
in the compound (X2), a total mass percentage of the oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 55% by mass or more based on a molecular weight of the compound (X2),
the heterocyclic compound (X3) has an imidazole skeleton or a pyridine skeleton and at least one of a hydroxy group or a carbonyl group, or has two or more skeletons selected from the group consisting of an imidazole skeleton and a pyridine skeleton,
the ketone (X4) has a linear β-diketone structure,
the substance (X) has a molecular weight of 550 or less,
the non-volatile component has an HLB value of 7 or more, and
the microbiota improving agent satisfies the following conditions (i) and (ii):
(i) the microbiota improving agent has a worked penetration of 100 to 400 as measured in accordance with JIS K 2220; and
(ii) the microbiota improving agent has a viscosity at 37° C. of 1 to 4,000 Pa·s as measured with a cone-and-plate rheometer.

The present disclosure (2) relates to the microbiota improving agent according to the present disclosure (1), wherein a solution having a concentration of 1% by weight of the microbiota improving agent prepared by diluting the microbiota improving agent with water has a pH of 6 to 8.

The present disclosure (3) relates to the microbiota improving agent according to the present disclosure (1) or (2),
wherein a weight percentage of the substance (X) is 0.01 to 5% by weight based on a weight of the microbiota improving agent.

### EXAMPLES

The present invention will be further described below with reference to examples and comparative examples, but the present invention is not limited thereto. In the following description, unless otherwise specified, “part(s)” means “part(s) by weight and “%” means “% by weight”.

### Examples 1 to 44 and Comparative Examples 1 to 16: Production of Microbiota Improving Agent

A substance (X) or (X′), a base (Y) or (Y′), and ion-exchanged water were mixed at 25° C. to obtain microbiota improving agents having the corresponding concentrations (% by weight) of these components shown in Tables 1-1 to 1-6.

The microbiota improving agents were subjected to measurements of viscosity and worked penetration by the methods described herein. The results are shown in Tables 1-1 to 1-6. Solutions having a concentration of 1% by weight of the microbiota improving agents were prepared by diluting the microbiota improving agents with water. The pHs of the solutions are also shown in Tables 1-1 to 1-6. The worked penetration of the microbiota improving agent of Comparative Example 14 exceeded 400 and was impossible to measure.

Solutions having a concentration of 1% by weight of the non-volatile components in the bases (Y) were prepared by dilution with ion-exchanged water. The viscosities of the solutions measured by the method described herein, and the HLB values and weight average molecular weights of the non-volatile components are shown in Table 2.

### <Description of Components>

The following describes the raw materials corresponding to the components used in the examples and comparative examples.

### Substance (X) or (X′)

(X-1): Pentasodium triphosphate (FUJIFILM Wako Pure Chemical Corporation, the total mass percentage of the oxoacid groups based on the molecular weight of triphosphoric acid: 88% by mass)
(X-2): Acetylacetone (Nacalai Tesque, Inc., the total mass percentage of the carbonyl groups based on the molecular weight of acetylacetone: 56% by mass)
(X-3): Benzoylacetone (MERCK, the total mass percentage of the carbonyl groups based on the molecular weight of benzoylacetone: 35% by mass)
(X-4): Levulinic acid (Tokyo Chemical Industry Co., Ltd., the total mass percentage of the oxoacid group and carbonyl group based on the molecular weight of levulinic acid: 63% by mass)
(X-5): Sodium tungstate dihydrate (Na₂WO₄·2H₂O available from FUJIFILM Wako Pure Chemical Corporation, the total mass percentage of the oxoacid group based on the molecular weight of tungstic acid: 100% by mass); the weight percentage of the substance (X) (Na₂WO₄) contained in (X-5) is 89% by weight based on the weight of (X-5)
(X-6): Glycylglycine (FUJIFILM Wako Pure Chemical Corporation, the total mass percentage of the oxoacid group and carbonyl group based on the molecular weight of glycylglycine: 55% by mass)
(X-7): Glutamic acid (FUJIFILM Wako Pure Chemical Corporation, the total mass percentage of the oxoacid groups based on the molecular weight of glutamic acid: 61% by mass)
(X-8): Malic acid (FUJIFILM Wako Pure Chemical Corporation, the total mass percentage of the oxoacid groups and hydroxy group based on the molecular weight of malic acid: 80% by mass)
(X-9): Citric acid (FUJIFILM Wako Pure Chemical Corporation, the total mass percentage of the oxoacid groups and hydroxy group based on the molecular weight of citric acid: 79% by mass)
(X-10): Gluconic acid (FUJIFILM Wako Pure Chemical Corporation, the total mass percentage of the oxoacid group and hydroxy groups based on the molecular weight of gluconic acid: 66% by mass)
(X-11): Glucuronic acid (FUJIFILM Wako Pure Chemical Corporation, the total mass percentage of the oxoacid group and hydroxy groups based on the molecular weight of glucuronic acid: 58% by mass]
(X-12): Ethylenediaminetetraacetic acid (FUJIFILM Wako Pure Chemical Corporation, the total mass percentage of the oxoacid groups based on the molecular weight of ethylenediaminetetraacetic acid: 62% by mass)
(X-13): Iminodiacetic acid (FUJIFILM Wako Pure Chemical Corporation, the total mass percentage of the oxoacid groups based on the molecular weight of iminodiacetic acid: 68% by mass)
(X-14): 8-Quinolinol (FUJIFILM Wako Pure Chemical Corporation, the total mass percentage of the carbon atoms and the nitrogen atom contained in the pyridine skeleton and the hydroxy group based on the molecular weight of 8-quinolinol: 63% by mass)
(X-15): Tartaric acid (FUJIFILM Wako Pure Chemical Corporation, the total mass percentage of the oxoacid groups and hydroxy groups based on the molecular weight of tartaric acid: 83% by mass)
(X-16): Adenosine triphosphate disodium trihydrate (FUJIFILM Wako Pure Chemical Corporation, the total mass percentage of the oxoacid groups, the hydroxy groups, and the carbon atoms and nitrogen atoms contained in the imidazole skeleton based on the molecular weight of adenosine triphosphate: 61% by mass); the weight percentage of the substance (X) (adenosine triphosphate disodium) contained in (X-16) is 91% by weight based on the weight of (X-16)
(X′-1): Benzalkonium chloride (FUJIFILM Wako Pure Chemical Corporation)
(X′-2): Cetylpyridinium chloride (FUJIFILM Wako Pure Chemical Corporation)

### Base (Y) or (Y′)

(Y-1): Carboxymethylethylcellulose (product name: CMEC, available from Sanyo Chemical Industries, Ltd.)
(Y-2): Sodium carboxymethylcellulose (FUJIFILM Wako Pure Chemical Corporation)
(Y-3): Sodium alginate (FUJIFILM Wako Pure Chemical Corporation)
(Y-4): Xanthan gum (FUJIFILM Wako Pure Chemical Corporation)
(Y-5): Starch-sodium acrylate graft copolymer (product name: Sunfresh ST100, available from Sanyo Chemical Industries, Ltd.)
(Y-6): PEG 20000 (Sanyo Chemical Industries, Ltd.)
(Y-7-1) to (Y-7-4): produced in Production Examples 1 to 4, respectively
(Y′-1): White petrolatum (FUJIFILM Wako Pure Chemical Corporation)
(Y-8): Methylcellulose 4000 (FUJIFILM Wako Pure Chemical Corporation)

Each substance (X) was subjected to measurement of the amount (g) of magnesium captured per 100 g of the substance (X) by the ion-selective electrode method as follows. For (X-5), the amount of magnesium captured per 100 g of Na₂WO₄ contained in (X-5) was measured. For (X-16), the amount of magnesium captured per 100 g of adenosine triphosphate disodium contained in (X-16) was measured.

The measurement by the ion-selective electrode method was performed using 2 mM of the substance (X) relative to a 1 mM aqueous MgCl₂ solution having a temperature of 20° C. and a pH of 7.

The results are shown in Table 3.

### Production Example 1: Production of Base (Y-7-1)

A planetary mixer PLM-15 equipped with a gate blade (Inoue Mfg., Inc.) was charged with 46.9 parts of polydimethylsiloxane (Dow Corning Toray Co., Ltd., Q7-9120 Silicone Fluid 1000 cSt (n=350), HLB value: 5.1) and 23.1 parts of an adduct of oxyethylene (15 mol) with cetyl alcohol (HLB value: 12.2). The contents were heated to 37° C., and then the temperature was maintained at 37° C. while the contents were stirred in the planetary mixer at a rotation speed of 20 rpm and a revolution speed of 50 rpm. To the mixer was gradually added 40 parts of warm water adjusted to 37° C. while checking the appearance. Thereby, a base (Y-7-1) was prepared.

### Production Example 2: Production of Base (Y-7-2)

A base (Y-7-2) was prepared as in Production Example 1, except that “630 parts of warm water” was used instead of “40 parts of warm water”.

### Production Example 3: Production of Base (Y-7-3)

A base (Y-7-3) was prepared as in Production Example 1, except that “3,430 parts of warm water” was used instead of “40 parts of warm water”.

### Production Example 4: Production of Base (Y-7-4)

A base (Y-7-4) was prepared as in Production Example 1, except that “20 parts of warm water” was used instead of “40 parts of warm water”.

### <Confirmation Test for Selective Inhibition of Bacterial Growth>

The pathogenic bacteria used in the test were Porphyromonas gingivalis, Fusobacterium nucleatum, Prevotella intermedia, Trichophyton rubrum, Trichophyton mentagrophytes, Staphylococcus aureus, Candida albicans, and Candida glabrata, while the non-pathogenic bacteria used were Streptococcus salivarius and Staphylococcus epidermidis.

These bacteria were cultured using the following media. A medium prepared by adding 1 μg/mL menadione to modified GAM Broth “Nissui” (Nissui Pharmaceutical Co., Ltd.) for Porphyromonas gingivalis; modified GAM Broth “Nissui” was used for Fusobacterium nucleatum, Prevotella intermedia, and Streptococcus salivarius; Sabouraud-dextrose broth (Nippon Pharmaceutical Co., Ltd.) was used for Trichophyton rubrum and Trichophyton mentagrophytes; LB medium (Becton Dickinson) was used for Staphylococcus aureus and Staphylococcus epidermidis; and YM medium (Sigma) was used for Candida albicans and Candida glabrata.

Porphyromonas gingivalis, Fusobacterium nucleatum, and Prevotella intermedia were cultured under anaerobic conditions, while Streptococcus salivarius, Trichophyton rubrum, Trichophyton mentagrophytes, Staphylococcus aureus, Staphylococcus epidermidis, Candida albicans, and Candida glabrata were cultured under aerobic conditions.

Regarding the microbiota improving agents prepared according to the formulations shown in Tables 1-1 to 1-6, 50 mg of any of the microbiota improving agents was added into each well of 24-well plates.

Separately, the bacterial strains were cultured in the corresponding media overnight at 37° C. under anaerobic conditions and diluted with the same culture media so that the dilutions had an absorbance at 600 nm of 0.2. Then, 500 μL of any of the dilutions was added into each well of a 24-well plate containing the microbiota improving agent, and static culture was performed at 37° C. under anaerobic conditions using AnaeroPack-Anaero 10% (Mitsubishi Gas Chemical Company, Inc.). Separately, the bacterial strains were cultured in the corresponding media overnight with shaking at 37° C. under aerobic conditions and diluted with the same culture media so that the dilutions had an absorbance at 600 nm of 0.2. Then, 500 μL of any of the dilutions was added into each well of a 24-well plate containing the microbiota improving agent, and static culture was performed at 37° C. After 48 hours, each plate was collected and the absorbance at 600 nm was measured to evaluate the growth characteristics of the bacteria.

The growth inhibitory effect was expressed as a relative value to the turbidity, which was taken as 1, in the case of using the same weight of ion-exchanged water instead of the microbiota improving agent. The results are shown in Tables 1-1 to 1-6.

In the case of applying the microbiota improving agent of the present invention to the oral cavity, when the growth characteristics of Porphyromonas gingivalis, Fusobacterium nucleatum, and Prevotella intermedia are more inhibited with the microbiota improving agent than without it, and the growth characteristics of Streptococcus salivarius remain unchanged from those without the microbiota improving agent, the bacterial growth is considered to be inhibited selectively.

For example, the relative values may fall within the following ranges.

The results of the tests for Porphyromonas gingivalis, Fusobacterium nucleatum, and Prevotella intermedia show that each of the relative values is 0.01 to 0.30, preferably 0.01 to 0.15, and the result of the test for Streptococcus salivarius shows that the relative value is 0.8 to 1.2.

In the case of applying the microbiota improving agent of the present invention to the skin and nail plates, when the growth characteristics of Trichophyton rubrum, Trichophyton mentagrophytes, Staphylococcus aureus, Candida albicans, and Candida glabrata are more inhibited with the microbiota improving agent than without it, and the growth characteristics of Staphylococcus epidermidis remain unchanged from those without the microbiota improving agent, the bacterial growth is considered to be inhibited selectively.

For example, the relative values may fall within the following ranges.

The results of the tests for Trichophyton rubrum, Trichophyton mentagrophytes, and Staphylococcus aureus show that each of the relative values are 0.01 to 0.30, preferably 0.01 to 0.15, and the result of the test for Staphylococcus epidermidis shows that the relative value is 0.8 to 1.2.

### <Assay of Inhibition of Protease Production by Pathogenic Bacteria>

The pathogenic bacteria used in the assay were Porphyromonas gingivalis, Fusobacterium nucleatum, and Prevotella intermedia. These bacteria were cultured using the following media. A medium prepared by adding 1 μg/mL menadione to modified GAM broth “Nissui” (Nissui Pharmaceutical Co., Ltd.) was used for Porphyromonas gingivalis, and modified GAM broth “Nissui” was used for Fusobacterium nucleatum and Prevotella intermedia.

Regarding the microbiota improving agents prepared according to the formulations shown in Tables 1-1 to 1-6, 50 mg of any of the microbiota improving agents was added into each well of a 24-well plate. Separately, the bacterial strains were cultured in the corresponding media overnight at 37° C. under anaerobic conditions and diluted with the same culture media so that the dilutions had an absorbance at 600 nm of 0.2. Then, 500 μL of any of the dilutions was added into each well of the 24-well plate containing the microbiota improving agent, and static culture was performed at 37° C. under anaerobic conditions using AnaeroPack-Anaero 10% (Mitsubishi Gas Chemical Company, Inc.). After 48 hours, the plate was collected and the culture was centrifuged at 4° C. and 10,000 rpm for five minutes to collect the culture supernatant. The activity of protease contained in the culture supernatant was evaluated using an Amplite protease activity assay kit (AAT Bioquest).

The protease production was expressed as a relative value to the protease activity value, which was taken as 1, in the case of using the same weight of ion-exchanged water instead of the microbiota improving agent. The results are shown in Tables 1-1 to 1-6.

Protease is known to be one of the toxin components produced by pathogenic bacteria. The lower the protease activity value, the more inhibited the protease production.

### <Evaluation of Persistence of Effect by Halo Test>

### Preparation of Agar Medium for Halo Test

The pathogenic bacteria used in the test were Porphyromonas gingivalis, Fusobacterium nucleatum, Prevotella intermedia, Trichophyton rubrum, Trichophyton mentagrophytes, Staphylococcus aureus, Candida albicans, and Candida glabrata, while the non-pathogenic bacteria used were Streptococcus salivarius and Staphylococcus epidermidis.

These bacteria were cultured using the following media. A medium prepared by adding 1 μg/mL menadione to modified GAM Broth “Nissui” (Nissui Pharmaceutical Co., Ltd.) was used for Porphyromonas gingivalis; modified GAM broth “Nissui” was used for Fusobacterium nucleatum, Prevotella intermedia, and Streptococcus salivarius; Sabouraud-dextrose broth (Nippon Pharmaceutical Co., Ltd.) was used for Trichophyton rubrum and Trichophyton mentagrophytes; LB medium (Becton Dickinson) was used for Staphylococcus aureus and Staphylococcus epidermidis; and YM medium (Sigma) was used for Candida albicans and Candida glabrata.

The bacterial strains were cultured in the corresponding media overnight at 37° C. under anaerobic or aerobic conditions and made into bacterial solutions of 1.0×10⁵ CFU/ml. Then, 100 μL of any of the bacterial solutions was inoculated on an agar medium prepared from any of the media and uniformly spread with a bacteria spreader.

### Halo Test

Regarding the microbiota improving agents prepared according to the formulations shown in Tables 1-1 to 1-6, 200 μL of any of the microbiota improving agents as a test sample was inoculated on the agar medium inoculated with any of the bacteria, followed by testing. The culture of bacteria under anaerobic conditions was performed at 37° C. for 24 hours using AnaeroPack-Anaero 10% (Mitsubishi Gas Chemical Company, Inc.). The culture of bacteria under aerobic conditions was performed at 37° C. for 24 hours. In the evaluation of the persistence of the growth inhibitory effect on pathogenic bacteria, for the agents rated as x (Poor) after 24 hours of culture, the culture of pathogenic bacteria was stopped, whereas for the other agents, the culture was continued at 37° C. for one week. For the agents rated as A (Fair) after one week of culture, the culture of pathogenic bacteria was stopped, whereas for the other agents, the culture was continued at 37° C. for two weeks. In the evaluation of the persistence of the effect on non-pathogenic bacteria, all the bacteria were cultured for two weeks.

The presence of a halo was determined by measuring the length from the edge of the test sample application area to the edge of a halo. When the length was greater than 0 mm, a halo was determined to be present, whereas when the length was 0 mm, a halo was determined to be absent.

In the assay of the growth inhibitory effect on pathogenic bacteria, the presence of a halo indicates having a growth inhibitory effect, whereas the absence of a halo indicates having no growth inhibitory effect. In the assay of the effect on non-pathogenic bacteria, the absence of a halo indicates no inhibition of the growth (growth is unaffected), whereas the presence of a halo indicates inhibition of the growth. The assay of the persistence of the growth inhibitory effect on pathogenic bacteria (persistence assay (pathogenic bacteria)) and the assay of the persistence of the effect on non-pathogenic bacteria (persistence assay (non-pathogenic bacteria)) were performed according to the following criteria. The evaluation results are shown in Tables 1-1 to 1-6.

### <Persistence Assay (Pathogenic Bacteria)>

◯◯ (Excellent): A halo was observed in the assays after 24 hours of culture, one week of culture, and two weeks of culture.

◯ (Good): A halo was observed in the assays after 24 hours of culture and one week of culture, but a halo was not observed (had disappeared) in the assay after two weeks of culture.

Δ (Fair): A halo was observed in the assay after 24 hours of culture, but was not observed in the assay after one week of culture.

x (Poor): No halo was observed in the assay after 24 hours of culture.

### <Persistence Assay (Non-Pathogenic Bacteria)>

◯◯ (Excellent): A halo was not observed even after 24 hours of culture, one week of culture, and two weeks of culture.

◯ (Good): A halo was observed in the assay after 24 hours of culture, but was not observed in the assays after one week of culture and two weeks of culture.

Δ (Fair): A halo was observed in the assays after 24 hours of culture and one week of culture, but was not observed in the assay after two weeks of culture.

x (Poor): A halo was observed in the assays after 24 hours of culture, one week of culture, and two weeks of culture.

### <Evaluation of Spreadability>

A 10 cm square piece of leather was immersed in 80 cc of artificial saliva (0.844 g/L sodium chloride, 1.2 g/L potassium chloride, 0.146 g/L calcium chloride hydrate, 0.052 g/L magnesium chloride, 0.342 g/L dipotassium phosphate) contained in a 15 cm diameter petri dish at 37° C. for 10 minutes. After 10 minutes, the moisture on the leather surface was removed with gauze. Sensory tests were conducted by 10 panelists as follows: 0.5 g of any of the microbiota improving agents was applied to the piece of leather with a finger; and the smoothness during the application and the amount of the microbiota improving agent remaining on the finger were evaluated. They were evaluated as follows and the results are shown in Tables 1-1 to 1-6.

◯◯ (Excellent): Seven or more panelists felt that the microbiota improving agent was smoothly applied and that no microbiota improving agent remained on their fingers.

◯ (Good): Seven or more panelists felt that the microbiota improving agent was smoothly applied but that the microbiota improving agent remained on their fingers.

Δ (Fair): Seven or more panelists felt that the agent was not smoothly applied and that no microbiota improving agent remained on their fingers.

x (Poor): Seven or more panelists felt that the microbiota improving agent was not smoothly applied and that the microbiota improving agent remained on their fingers.

TABLE 1-1

| | | | Example | | | | |
|---|---|---|---|---|---|---|---|
| | | | 1 | 2 | 3 | 4 | 5 |
| Substance (X) or (X′) | | X-1 | 0.05 | 1.0 | 3.0 | 3.0 | 3.0 |
| | | X-2 | — | — | — | — | — |
| | | X-3 | — | — | — | — | — |
| | | X-4 | — | — | — | — | — |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | — | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | 2.0 | 2.0 | 2.0 | — | — |
| | | Y-2 | — | — | — | 2.0 | — |
| | | Y-3 | — | — | — | — | — |
| | | Y-4 | — | — | — | — | 5.0 |
| | | Y-5 | — | — | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 97.95 | 97.00 | 95.00 | 95.00 | 92.00 |
| Viscosity of microbiota improving agent (Pa · s) | | | 5.5 | 5.5 | 5.5 | 13.1 | 18.1 |
| Worked penetration of microbiota improving agent | | | 290 | 286 | 292 | 279 | 280 |
| pH of solution having concentration of 1% by weight of | | | 7.2 | 7.1 | 6.9 | 7.0 | 7.2 |
| microbiota improving agent prepared by dilution with water | | | | | | | |
| Confirmation | Pathogenic | Porphyromonas gingivalis | 0.1 | 0.07 | 0.04 | 0.05 | 0.05 |
| test for | bacteria | Fusobacterium nucleatum | 0.11 | 0.08 | 0.04 | 0.05 | 0.05 |
| selective | | Prevotella intermedia | 0.09 | 0.07 | 0.05 | 0.05 | 0.05 |
| inhibition of | | Trichophyton rubrum | 0.08 | 0.07 | 0.05 | 0.04 | 0.05 |
| bacterial | | Trichophyton | 0.09 | 0.07 | 0.05 | 0.05 | 0.05 |
| growth | | Staphylococcus aureus | 0.09 | 0.07 | 0.05 | 0.05 | 0.05 |
| | | Candida albicans | 0.2 | 0.09 | 0.05 | 0.05 | 0.05 |
| | | Candida glabrata | 0.18 | 0.06 | 0.05 | 0.05 | 0.05 |
| | Non- | Streptococcus salivarius | 0.9 | 1.0 | 1.0 | 1.1 | 0.9 |
| | pathogenic | Staphylococcus epidermidis | 1.0 | 1.1 | 0.9 | 1.0 | 1.0 |
| | bacteria | | | | | | |
| Inhibition assay of | | Porphyromonas gingivalis | 0.001 | 0.001 | 0.001 | 0.001 | 0.001 |
| protease production by | | Fusobacterium nucleatum | 0.002 | 0.001 | 0.001 | 0.001 | 0.001 |
| pathogenic bacteria | | Prevotella intermedia | 0.001 | 0.001 | 0.002 | 0.001 | 0.001 |
| Persistence | Pathogenic | Porphyromonas gingivalis | Δ | Δ | Δ | ∘ | ∘ |
| evaluation | bacteria | Fusobacterium nucleatum | Δ | Δ | Δ | ∘ | ∘ |
| | | Prevotella intermedia | Δ | Δ | Δ | ∘ | ∘ |
| | | Trichophyton rubrum | Δ | Δ | Δ | ∘ | ∘ |
| | | Trichophyton | Δ | Δ | Δ | ∘ | ∘ |
| | | Staphylococcus aureus | Δ | Δ | Δ | ∘ | ∘ |
| | | Candida albicans | Δ | Δ | Δ | ∘ | ∘ |
| | | Candida glabrata | Δ | Δ | Δ | ∘ | ∘ |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |
| Spreadability evaluation | | | ∘ | ∘ | ∘ | ∘ | ∘ |
| | | | Example | | | | |
| | | | 6 | 7 | 8 | 9 | 10 |
| Substance (X) or (X′) | | X-1 | 3.0 | 3.0 | — | — | — |
| | | X-2 | — | — | 3.0 | — | — |
| | | X-3 | — | — | — | 3.0 | — |
| | | X-4 | — | — | — | — | 3.0 |
| | | X-5 | — | — | — | — | — |
| | | X-6 | — | — | — | — | — |
| | | X-7 | — | — | — | — | — |
| | | X-8 | — | — | — | — | — |
| | | X-9 | — | 0.35 | — | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | — |
| | | Y-2 | — | — | — | — | — |
| | | Y-3 | — | — | — | — | — |
| | | Y-4 | 5.0 | 5.0 | 5.0 | 5.0 | 5.0 |
| | | Y-5 | — | — | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | 10.0 | — | — | — | — |
| Volatile component | | Ion-exchanged water | 82.00 | 91.65 | 92.00 | 92.00 | 92.00 |
| Viscosity of microbiota improving agent (Pa · s) | | | 20 | 18.1 | 18.2 | 17.9 | 18.1 |
| Worked penetration of microbiota improving agent | | | 250 | 280 | 275 | 279 | 280 |
| pH of solution having concentration of 1% by weight of | | | 7.2 | 7.2 | 7.1 | 7.2 | 6.9 |
| microbiota improving agent prepared by dilution with water | | | | | | | |
| Confirmation | Pathogenic | Porphyromonas gingivalis | 0.05 | 0.05 | 0.06 | 0.05 | 0.05 |
| test for | bacteria | Fusobacterium nucleatum | 0.05 | 0.05 | 0.05 | 0.05 | 0.05 |
| selective | | Prevotella intermedia | 0.05 | 0.05 | 0.05 | 0.06 | 0.05 |
| inhibition of | | Trichophyton rubrum | 0.05 | 0.05 | 0.05 | 0.06 | 0.05 |
| bacterial | | Trichophyton | 0.05 | 0.05 | 0.05 | 0.07 | 0.05 |
| growth | | Staphylococcus aureus | 0.05 | 0.05 | 0.05 | 0.06 | 0.05 |
| | | Candida albicans | 0.05 | 0.05 | 0.05 | 0.05 | 0.05 |
| | | Candida glabrata | 0.05 | 0.05 | 0.05 | 0.05 | 0.05 |
| | Non- | Streptococcus salivarius | 0.9 | 0.9 | 0.9 | 1.0 | 1.0 |
| | pathogenic | Staphylococcus epidermidis | 0.9 | 1.0 | 0.9 | 1.0 | 0.9 |
| | bacteria | | | | | | |
| Inhibition assay of | | Porphyromonas gingivalis | 0.001 | 0.001 | 0.001 | 0.001 | 0.001 |
| protease production by | | Fusobacterium nucleatum | 0.001 | 0.001 | 0.001 | 0.002 | 0.001 |
| pathogenic bacteria | | Prevotella intermedia | 0.001 | 0.001 | 0.001 | 0.001 | 0.001 |
| Persistence | Pathogenic | Porphyromonas gingivalis | ∘∘ | ∘ | ∘ | ∘ | ∘ |
| evaluation | bacteria | Fusobacterium nucleatum | ∘∘ | ∘ | ∘ | ∘ | ∘ |
| | | Prevotella intermedia | ∘∘ | ∘ | ∘ | ∘ | ∘ |
| | | Trichophyton rubrum | ∘∘ | ∘ | ∘ | ∘ | ∘ |
| | | Trichophyton | ∘∘ | ∘ | ∘ | ∘ | ∘ |
| | | Staphylococcus aureus | ∘∘ | ∘ | ∘ | ∘ | ∘ |
| | | Candida albicans | ∘∘ | ∘ | ∘ | ∘ | ∘ |
| | | Candida glabrata | ∘∘ | ∘ | ∘ | ∘ | ∘ |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |

| | | | | | |
|---|---|---|---|---|---|
| Spreadability evaluation | ∘ | ∘ | ∘ | ∘ | ∘ |

TABLE 1-2

| | | | Example | | | | |
|---|---|---|---|---|---|---|---|
| | | | 11 | 12 | 13 | 14 | 15 |
| Substance (X) or (X′) | | X-1 | — | — | — | — | — |
| | | X-2 | — | — | — | — | — |
| | | X-3 | — | — | — | — | — |
| | | X-4 | — | — | — | — | — |
| | | X-5 | 3.0 | — | — | — | — |
| | | X-6 | — | 3.0 | — | — | — |
| | | X-7 | — | — | 3.0 | — | — |
| | | X-8 | — | — | — | 3.0 | — |
| | | X-9 | — | — | — | — | 3.0 |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | — |
| | | Y-2 | — | — | — | — | — |
| | | Y-3 | — | — | — | — | — |
| | | Y-4 | 5.0 | 5.0 | 5.0 | 5.0 | 5.0 |
| | | Y-5 | — | — | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 92.00 | 92.00 | 92.00 | 92.00 | 92.00 |
| Viscosity of microbiota improving agent (Pa · s) | | | 17.7 | 18.2 | 18.0 | 17.7 | 17.5 |
| Worked penetration of microbiota improving agent | | | 278 | 275 | 278 | 278 | 277 |
| pH of solution having concentration of 1% by weight of | | | 7.0 | 7.0 | 6.9 | 7.0 | 6.4 |
| microbiota improving agent prepared by dilution with water | | | | | | | |
| Confirmation | Pathogenic | Porphyromonas gingivalis | 0.05 | 0.05 | 0.06 | 0.05 | 0.1 |
| test for | bacteria | Fusobacterium nucleatum | 0.05 | 0.05 | 0.05 | 0.06 | 0.09 |
| selective | | Prevotella intermedia | 0.06 | 0.06 | 0.06 | 0.06 | 0.08 |
| inhibition of | | Trichophyton rubrum | 0.05 | 0.05 | 0.05 | 0.05 | 0.1 |
| bacterial | | Trichophyton mentagrophytes | 0.06 | 0.06 | 0.05 | 0.06 | 0.11 |
| growth | | Staphylococcus aureus | 0.06 | 0.06 | 0.06 | 0.05 | 0.1 |
| | | Candida albicans | 0.05 | 0.05 | 0.05 | 0.07 | 0.2 |
| | | Candida glabrata | 0.05 | 0.05 | 0.05 | 0.05 | 0.15 |
| | Non- | Streptococcus salivarius | 1.1 | 1.1 | 1.0 | 0.9 | 1.1 |
| | pathogenic | Staphylococcus epidermidis | 1.0 | 1.0 | 1.0 | 1.0 | 0.9 |
| | bacteria | | | | | | |
| Inhibition assay of | | Porphyromonas gingivalis | 0.001 | 0.001 | 0.001 | 0.001 | 0.003 |
| protease production by | | Fusobacterium nucleatum | 0.002 | 0.002 | 0.002 | 0.002 | 0.002 |
| pathogenic bacteria | | Prevotella intermedia | 0.001 | 0.001 | 0.001 | 0.001 | 0.001 |
| Persistence | Pathogenic | Porphyromonas gingivalis | ∘ | ∘ | ∘ | ∘ | Δ |
| evaluation | bacteria | Fusobacterium nucleatum | ∘ | ∘ | ∘ | ∘ | Δ |
| | | Prevotella intermedia | ∘ | ∘ | ∘ | ∘ | Δ |
| | | Trichophyton rubrum | ∘ | ∘ | ∘ | ∘ | Δ |
| | | Trichophyton mentagrophytes | ∘ | ∘ | ∘ | ∘ | Δ |
| | | Staphylococcus aureus | ∘ | ∘ | ∘ | ∘ | Δ |
| | | Candida albicans | ∘ | ∘ | ∘ | ∘ | Δ |
| | | Candida glabrata | ∘ | ∘ | ∘ | ∘ | Δ |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |
| Spreadability evaluation | | | ∘ | ∘ | ∘ | ∘ | ∘ |
| | | | Example | | | | |
| | | | 16 | 17 | 18 | 19 | 20 |
| Substance (X) or (X′) | | X-1 | — | — | — | — | — |
| | | X-2 | — | — | — | — | — |
| | | X-3 | — | — | — | — | — |
| | | X-4 | — | — | — | — | — |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | — | — | — |
| | | X-10 | 3.0 | — | — | — | — |
| | | X-11 | — | 3.0 | — | — | — |
| | | X-12 | — | — | 3.0 | — | — |
| | | X-13 | — | — | — | 3.0 | — |
| | | X-14 | — | — | — | — | 3.0 |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | — |
| | | Y-2 | — | — | — | — | — |
| | | Y-3 | — | — | — | — | — |
| | | Y-4 | 5.0 | 5.0 | 5.0 | 5.0 | 5.0 |
| | | Y-5 | — | — | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 92.00 | 92.00 | 92.00 | 92.00 | 92.00 |
| Viscosity of microbiota improving agent (Pa · s) | | | 18.1 | 18.1 | 17.7 | 17.7 | 17.9 |
| Worked penetration of microbiota improving agent | | | 274 | 274 | 278 | 278 | 278 |
| pH of solution having concentration of 1% by weight of | | | 6.5 | 7.0 | 7.5 | 7.0 | 7.2 |
| microbiota improving agent prepared by dilution with water | | | | | | | |
| Confirmation | Pathogenic | Porphyromonas gingivalis | 0.05 | 0.05 | 0.05 | 0.05 | 0.05 |
| test for | bacteria | Fusobacterium nucleatum | 0.05 | 0.05 | 0.06 | 0.05 | 0.06 |
| selective | | Prevotella intermedia | 0.06 | 0.06 | 0.06 | 0.05 | 0.06 |
| inhibition of | | Trichophyton rubrum | 0.06 | 0.05 | 0.05 | 0.05 | 0.05 |
| bacterial | | Trichophyton mentagrophytes | 0.06 | 0.06 | 0.06 | 0.06 | 0.06 |
| growth | | Staphylococcus aureus | 0.06 | 0.06 | 0.06 | 0.06 | 0.06 |
| | | Candida albicans | 0.05 | 0.05 | 0.05 | 0.06 | 0.05 |
| | | Candida glabrata | 0.06 | 0.05 | 0.05 | 0.05 | 0.05 |
| | Non- | Streptococcus salivarius | 1.1 | 1.0 | 1.1 | 0.9 | 1.1 |
| | pathogenic | Staphylococcus epidermidis | 1.0 | 1.0 | 1.0 | 1.0 | 0.9 |
| | bacteria | | | | | | |
| Inhibition assay of | | Porphyromonas gingivalis | 0.001 | 0.001 | 0.001 | 0.001 | 0.001 |
| protease production by | | Fusobacterium nucleatum | 0.002 | 0.002 | 0.002 | 0.002 | 0.002 |
| pathogenic bacteria | | Prevotella intermedia | 0.001 | 0.001 | 0.001 | 0.001 | 0.001 |
| Persistence | Pathogenic | Porphyromonas gingivalis | ∘ | ∘ | ∘ | ∘ | ∘ |
| evaluation | bacteria | Fusobacterium nucleatum | ∘ | ∘ | ∘ | ∘ | ∘ |
| | | Prevotella intermedia | ∘ | ∘ | ∘ | ∘ | ∘ |
| | | Trichophyton rubrum | ∘ | ∘ | ∘ | ∘ | ∘ |
| | | Trichophyton mentagrophytes | ∘ | ∘ | ∘ | ∘ | ∘ |
| | | Staphylococcus aureus | ∘ | ∘ | ∘ | ∘ | ∘ |
| | | Candida albicans | ∘ | ∘ | ∘ | ∘ | ∘ |
| | | Candida glabrata | ∘ | ∘ | ∘ | ∘ | ∘ |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |

| | | | | | |
|---|---|---|---|---|---|
| Spreadability evaluation | ∘ | ∘ | ∘ | ∘ | ∘ |

TABLE 1-3

| | | | Example | | | | |
|---|---|---|---|---|---|---|---|
| | | | 21 | 22 | 23 | 24 | 25 |
| Substance (X) or (X′) | | X-1 | — | — | — | 3.0 | 3.0 |
| | | X-2 | — | — | — | — | — |
| | | X-3 | — | — | — | — | — |
| | | X-4 | — | — | — | — | — |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | 1.0 | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | 3.0 | — | — | — | — |
| | | X-16 | — | 3.0 | — | — | — |
| | | X′-1 | — | — | — | — | — |
| | | X′-2 | — | — | — | 0.05 | — |
| Base (Y) or (Y′) | | Y-1 | — | — | — | — | — |
| | | Y-2 | — | — | — | — | — |
| | | Y-3 | — | — | — | — | 10.0 |
| | | Y-4 | 5.0 | 5.0 | 5.0 | 5.0 | — |
| | | Y-5 | — | — | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 92.00 | 92.00 | 94.00 | 91.95 | 87.00 |
| Viscosity of microbiota improving agent (Pa · s) | | | 18.0 | 17.9 | 17.0 | 18.1 | 20 |
| Worked penetration of microbiota improving agent | | | 280 | 282 | 285 | 280 | 270 |
| pH of solution having concentration of 1% by weight of | | | 7.1 | 7.1 | 6.5 | 7.2 | 7.1 |
| microbiota improving agent prepared by dilution with water | | | | | | | |
| Confirmation | Pathogenic | Porphyromonas gingivalis | 0.05 | 0.07 | 0.31 | 0.05 | 0.05 |
| test for | bacteria | Fusobacterium nucleatum | 0.05 | 0.09 | 0.22 | 0.05 | 0.05 |
| selective | | Prevotella intermedia | 0.06 | 0.06 | 0.17 | 0.05 | 0.06 |
| inhibition of | | Trichophyton rubrum | 0.05 | 0.07 | 0.22 | 0.05 | 0.06 |
| bacterial | | Trichophyton mentagrophytes | 0.06 | 0.11 | 0.24 | 0.05 | 0.06 |
| growth | | Staphylococcus aureus | 0.06 | 0.21 | 0.31 | 0.05 | 0.06 |
| | | Candida albicans | 0.07 | 0.09 | 0.41 | 0.05 | 0.05 |
| | | Candida glabrata | 0.05 | 0.10 | 0.21 | 0.05 | 0.05 |
| | Non- | Streptococcus salivarius | 1.0 | 1.0 | 1.0 | 0.1 | 1.0 |
| | pathogenic | Staphylococcus epidermidis | 1.0 | 1.0 | 1.0 | 0.1 | 1.0 |
| | bacteria | | | | | | |
| Inhibition assay of | | Porphyromonas gingivalis | 0.001 | 0.001 | 0.01 | 0.001 | 0.001 |
| protease production by | | Fusobacterium nucleatum | 0.002 | 0.001 | 0.003 | 0.001 | 0.002 |
| pathogenic bacteria | | Prevotella intermedia | 0.001 | 0.001 | 0.002 | 0.001 | 0.001 |
| Persistence | Pathogenic | Porphyromonas gingivalis | ∘ | Δ | Δ | ∘ | ∘ |
| evaluation | bacteria | Fusobacterium nucleatum | ∘ | Δ | Δ | ∘ | ∘ |
| | | Prevotella intermedia | ∘ | Δ | Δ | ∘ | ∘ |
| | | Trichophyton rubrum | ∘ | Δ | Δ | ∘ | ∘ |
| | | Trichophyton mentagrophytes | ∘ | Δ | Δ | ∘ | ∘ |
| | | Staphylococcus aureus | ∘ | Δ | Δ | ∘ | ∘ |
| | | Candida albicans | ∘ | Δ | Δ | ∘ | ∘ |
| | | Candida glabrata | ∘ | Δ | Δ | ∘ | ∘ |
| | Non- | Streptococcus salivarius | ∘∘ | ∘∘ | ∘∘ | ∘ | ∘∘ |
| | pathogenic | Staphylococcus epidermidis | ∘∘ | ∘∘ | ∘∘ | ∘ | ∘∘ |
| | bacteria | | | | | | |
| Spreadability evaluation | | | ∘ | ∘ | ∘ | ∘ | ∘ |
| | | | Example | | | | |
| | | | 26 | 27 | 28 | 29 | 30 |
| Substance (X) or (X′) | | X-1 | 3.0 | 3.0 | — | — | — |
| | | X-2 | — | — | 3.0 | — | — |
| | | X-3 | — | — | — | 3.0 | |
| | | X-4 | — | — | — | — | 3.0 |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | — | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | — |
| | | Y-2 | — | — | — | — | — |
| | | Y-3 | — | — | 10.0 | 10.0 | 10.0 |
| | | Y-4 | 10.0 | — | — | — | — |
| | | Y-5 | — | 10.0 | — | — | — |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 87.00 | 87.00 | 87.00 | 87.00 | 87.00 |
| Viscosity of microbiota improving agent (Pa · s) | | | 19.3 | 42 | 20 | 20 | 20 |
| Worked penetration of microbiota improving agent | | | 274 | 260 | 251 | 249 | 250 |
| pH of solution having concentration of 1% by weight of | | | 7.2 | 6.8 | 7.1 | 7.2 | 6.9 |
| microbiota improving agent prepared by dilution with water | | | | | | | |
| Confirmation | Pathogenic | Porphyromonas gingivalis | 0.05 | 0.05 | 0.06 | 0.05 | 0.05 |
| test for | bacteria | Fusobacterium nucleatum | 0.05 | 0.05 | 0.05 | 0.05 | 0.05 |
| selective | | Prevotella intermedia | 0.05 | 0.05 | 0.05 | 0.06 | 0.05 |
| inhibition of | | Trichophyton rubrum | 0.05 | 0.05 | 0.05 | 0.06 | 0.05 |
| bacterial | | Trichophyton mentagrophytes | 0.05 | 0.05 | 0.05 | 0.07 | 0.05 |
| growth | | Staphylococcus aureus | 0.05 | 0.05 | 0.05 | 0.06 | 0.05 |
| | | Candida albicans | 0.05 | 0.05 | 0.05 | 0.05 | 0.05 |
| | | Candida glabrata | 0.05 | 0.05 | 0.05 | 0.05 | 0.05 |
| | Non- | Streptococcus salivarius | 0.9 | 1.0 | 0.9 | 1.0 | 1.0 |
| | pathogenic | Staphylococcus epidermidis | 1.0 | 1.0 | 0.9 | 1.0 | 0.9 |
| | bacteria | | | | | | |
| Inhibition assay of | | Porphyromonas gingivalis | 0.001 | 0.001 | 0.001 | 0.001 | 0.001 |
| protease production by | | Fusobacterium nucleatum | 0.001 | 0.001 | 0.001 | 0.002 | 0.001 |
| pathogenic bacteria | | Prevotella intermedia | 0.001 | 0.001 | 0.001 | 0.001 | 0.001 |
| Persistence | Pathogenic | Porphyromonas gingivalis | ∘ | ∘ | ∘ | ∘ | ∘ |
| evaluation | bacteria | Fusobacterium nucleatum | ∘ | ∘ | ∘ | ∘ | ∘ |
| | | Prevotella intermedia | ∘ | ∘ | ∘ | ∘ | ∘ |
| | | Trichophyton rubrum | ∘ | ∘ | ∘ | ∘ | ∘ |
| | | Trichophyton mentagrophytes | ∘ | ∘ | ∘ | ∘ | ∘ |
| | | Staphylococcus aureus | ∘ | ∘ | ∘ | ∘ | ∘ |
| | | Candida albicans | ∘ | ∘ | ∘ | ∘ | ∘ |
| | | Candida glabrata | ∘ | ∘ | ∘ | ∘ | ∘ |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |

| | | | | | |
|---|---|---|---|---|---|
| Spreadability evaluation | ∘ | ∘ | ∘ | ∘ | ∘ |

TABLE 1-4

| | | | Example | | | | | |
|---|---|---|---|---|---|---|---|---|
| | | | 31 | 32 | 33 | 34 | 35 | 36 |
| Substance (X) or (X′) | | X-1 | — | 3.0 | 3.0 | 3.0 | 3.0 | 3.0 |
| | | X-2 | — | — | — | — | — | — |
| | | X-3 | — | — | — | — | — | — |
| | | X-4 | — | — | — | — | — | — |
| | | X-5 | 3.0 | — | — | — | — | — |
| | | X-6 | — | — | — | — | — | — |
| | | X-7 | — | — | — | — | — | — |
| | | X-8 | — | — | — | — | — | — |
| | | X-9 | — | — | — | — | — | — |
| | | X-10 | — | — | — | — | — | — |
| | | X-11 | — | — | — | — | — | — |
| | | X-12 | — | — | — | — | — | — |
| | | X-13 | — | — | — | — | — | — |
| | | X-14 | — | — | — | — | — | — |
| | | X-15 | — | — | — | — | — | — |
| | | X-16 | — | — | — | — | — | — |
| | | X′-1 | — | — | — | — | — | — |
| | | X′-2 | — | — | — | — | — | — |
| Base (Y) or (Y′) | | Y-1 | — | — | — | — | — | — |
| | | Y-2 | — | — | — | — | — | — |
| | | Y-3 | 10.0 | — | — | — | — | — |
| | | Y-4 | — | — | — | — | — | 0.1 |
| | | Y-5 | — | — | — | — | — | — |
| | | Y-6 | — | 50.0 | — | — | — | — |
| | | Y-7-1 | — | — | 97.0 | — | — | 96.9 |
| | | Y-7-2 | — | — | — | 97.0 | — | — |
| | | Y-7-3 | — | — | — | | 97.0 | — |
| | | Y′-1 | — | — | — | — | — | — |
| | | Y-7-4 | — | — | — | — | — | — |
| | | Y-8 | — | — | — | — | — | — |
| Volatile component | | Ion-exchanged water | 87.00 | 47.00 | — | — | — | — |
| Viscosity of microbiota improving agent (Pa · s) | | | 20 | 2.2 | 3500 | 90 | 8.9 | 3400 |
| Worked penetration of microbiota improving agent | | | 251 | 298 | 250 | 301 | 390 | 249 |
| pH of solution having concentration of 1% by weight of | | | 7.0 | 7.1 | 6.5 | 6.8 | 7.0 | 6.6 |
| microbiota improving agent prepared by dilution with water | | | | | | | | |
| Confirmation | Pathogenic | Porphyromonas gingivalis | 0.05 | 0.06 | 0.05 | 0.05 | 0.05 | 0.05 |
| test for | bacteria | Fusobacterium nucleatum | 0.05 | 0.05 | 0.05 | 0.05 | 0.05 | 0.05 |
| selective | | Prevotella intermedia | 0.06 | 0.07 | 0.05 | 0.05 | 0.05 | 0.06 |
| inhibition of | | Trichophyton rubrum | 0.05 | 0.07 | 0.05 | 0.05 | 0.05 | 0.05 |
| bacterial | | Trichophyton | 0.06 | 0.05 | 0.05 | 0.06 | 0.05 | 0.06 |
| growth | | Staphylococcus aureus | 0.06 | 0.07 | 0.06 | 0.05 | 0.05 | 0.06 |
| | | Candida albicans | 0.05 | 0.07 | 0.05 | 0.05 | 0.07 | 0.05 |
| | | Candida glabrata | 0.05 | 0.07 | 0.05 | 0.05 | 0.08 | 0.05 |
| | Non- | Streptococcus salivarius | 1.1 | 0.9 | 1.1 | 1.0 | 1.0 | 1.1 |
| | pathogenic | Staphylococcus epidermidis | 1.0 | 1.0 | 0.9 | 1.0 | 0.9 | 1.0 |
| | bacteria | | | | | | | |
| Inhibition assay of | | Porphyromonas gingivalis | 0.001 | 0.002 | 0.001 | 0.001 | 0.001 | 0.001 |
| protease production by | | Fusobacterium nucleatum | 0.002 | 0.001 | 0.001 | 0.001 | 0.001 | 0.002 |
| pathogenic bacteria | | Prevotella intermedia | 0.001 | 0.001 | 0.001 | 0.001 | 0.002 | 0.001 |
| Persistence | Pathogenic | Porphyromonas gingivalis | ∘ | Δ | ∘ | ∘ | Δ | ∘∘ |
| evaluation | bacteria | Fusobacterium nucleatum | ∘ | Δ | ∘ | ∘ | Δ | ∘∘ |
| | | Prevotella intermedia | ∘ | Δ | ∘ | ∘ | Δ | ∘∘ |
| | | Trichophyton rubrum | ∘ | Δ | ∘ | ∘ | Δ | ∘∘ |
| | | Trichophyton | ∘ | Δ | ∘ | ∘ | Δ | ∘∘ |
| | | Staphylococcus aureus | ∘ | Δ | ∘ | ∘ | Δ | ∘∘ |
| | | Candida albicans | ∘ | Δ | ∘ | ∘ | Δ | ∘∘ |
| | | Candida glabrata | ∘ | Δ | ∘ | ∘ | Δ | ∘∘ |
| | Non- | Streptococcus salivarius | ∘∘ | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ | ∘∘ |
| | bacteria | | | | | | | |

| | | | | | | |
|---|---|---|---|---|---|---|
| Spreadability evaluation | ∘ | ∘ | ∘∘ | ∘ | ∘ | ∘∘ |

| | | | Example | | | |
|---|---|---|---|---|---|---|
| | | | 37 | 38 | 39 | 40 |
| Substance (X) or (X′) | | X-1 | 3.0 | 3.0 | 3.0 | 3.0 |
| | | X-2 | — | — | — | — |
| | | X-3 | — | — | — | — |
| | | X-4 | — | — | — | — |
| | | X-5 | — | — | — | — |
| | | X-6 | — | — | — | — |
| | | X-7 | — | — | — | — |
| | | X-8 | — | — | — | — |
| | | X-9 | — | — | — | — |
| | | X-10 | — | — | — | — |
| | | X-11 | — | — | — | — |
| | | X-12 | — | — | — | — |
| | | X-13 | — | — | — | — |
| | | X-14 | — | — | — | — |
| | | X-15 | — | — | — | — |
| | | X-16 | — | — | — | — |
| | | X′-1 | — | — | — | — |
| | | X′-2 | — | — | — | — |
| Base (Y) or (Y′) | | Y-1 | — | — | — | — |
| | | Y-2 | — | — | 1.0 | — |
| | | Y-3 | — | — | — | — |
| | | Y-4 | 1.0 | 3.0 | — | 0.1 |
| | | Y-5 | — | — | — | — |
| | | Y-6 | — | — | — | — |
| | | Y-7-1 | 96.0 | 94.0 | 96.0 | — |
| | | Y-7-2 | — | — | — | — |
| | | Y-7-3 | — | — | — | 96.9 |
| | | Y′-1 | — | — | — | — |
| | | Y-7-4 | — | — | — | — |
| | | Y-8 | — | — | — | — |
| Volatile component | | Ion-exchanged water | — | — | — | — |
| Viscosity of microbiota improving agent (Pa · s) | | | 3650 | 3900 | 3600 | 8.6 |
| Worked penetration of microbiota improving agent | | | 239 | 101 | 260 | 392 |
| pH of solution having concentration of 1% by weight of | | | 6.9 | 7.0 | 6.8 | 7.0 |
| microbiota improving agent prepared by dilution with water | | | | | | |
| Confirmation | Pathogenic | Porphyromonas gingivalis | 0.03 | 0.05 | 0.04 | 0.06 |
| test for | bacteria | Fusobacterium nucleatum | 0.04 | 0.05 | 0.05 | 0.05 |
| selective | | Prevotella intermedia | 0.03 | 0.06 | 0.06 | 0.06 |
| inhibition of | | Trichophyton rubrum | 0.02 | 0.05 | 0.03 | 0.07 |
| bacterial | | Trichophyton | 0.03 | 0.06 | 0.04 | 0.06 |
| growth | | Staphylococcus aureus | 0.03 | 0.06 | 0.04 | 0.05 |
| | | Candida albicans | 0.05 | 0.07 | 0.05 | 0.05 |
| | | Candida glabrata | 0.05 | 0.08 | 0.05 | 0.05 |
| | Non- | Streptococcus salivarius | 1.1 | 1.1 | 1.0 | 1.0 |
| | pathogenic | Staphylococcus epidermidis | 1.0 | 1.0 | 0.9 | 1.0 |
| | bacteria | | | | | |
| Inhibition assay of | | Porphyromonas gingivalis | 0.001 | 0.001 | 0.001 | 0.001 |
| protease production by | | Fusobacterium nucleatum | 0.001 | 0.002 | 0.001 | 0.002 |
| pathogenic bacteria | | Prevotella intermedia | 0.001 | 0.001 | 0.001 | 0.001 |
| Persistence | Pathogenic | Porphyromonas gingivalis | ∘∘ | ∘∘ | ∘∘ | ∘∘ |
| evaluation | bacteria | Fusobacterium nucleatum | ∘∘ | ∘∘ | ∘∘ | ∘∘ |
| | | Prevotella intermedia | ∘∘ | ∘∘ | ∘∘ | ∘∘ |
| | | Trichophyton rubrum | ∘∘ | ∘∘ | ∘∘ | ∘∘ |
| | | Trichophyton | ∘∘ | ∘∘ | ∘∘ | ∘∘ |
| | | Staphylococcus aureus | ∘∘ | ∘∘ | ∘∘ | ∘∘ |
| | | Candida albicans | ∘∘ | ∘∘ | ∘∘ | ∘∘ |
| | | Candida glabrata | ∘∘ | ∘∘ | ∘∘ | ∘∘ |
| | Non- | Streptococcus salivarius | ∘∘ | ∘∘ | ∘∘ | ∘∘ |
| | pathogenic | Staphylococcus epidermidis | ∘∘ | ∘∘ | ∘∘ | ∘∘ |
| | bacteria | | | | | |

| | | | | |
|---|---|---|---|---|
| Spreadability evaluation | ∘∘ | ∘ | ∘∘ | ∘ |

TABLE 1-5

| | | | Example | | | | Comparative Example |
|---|---|---|---|---|---|---|---|
| | | | 41 | 42 | 43 | 44 | 1 |
| Substance (X) or (X′) | | X-1 | 3.0 | 3.0 | 3.0 | 3.0 | — |
| | | X-2 | — | — | — | — | — |
| | | X-3 | — | — | — | — | — |
| | | X-4 | — | — | — | — | — |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | — | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | 2.0 |
| | | Y-2 | — | — | — | — | — |
| | | Y-3 | 0.1 | 1.0 | 5.0 | 0.1 | — |
| | | Y-4 | — | — | — | — | — |
| | | Y-5 | — | — | — | — | — |
| | | Y-6 | — | — | — | — | — |
| | | Y-7-1 | 96.9 | 96.0 | 92.0 | — | — |
| | | Y-7-2 | — | — | — | — | — |
| | | Y-7-3 | — | — | — | 96.9 | — |
| | | Y′-1 | — | — | — | — | — |
| | | Y-7-4 | — | — | — | — | — |
| | | Y-8 | — | — | — | — | — |
| Volatile component | | Ion-exchanged water | — | — | — | — | 98.00 |
| Viscosity of microbiota improving agent (Pa · s) | | | 3400 | 3650 | 3900 | 9.0 | 5.5 |
| Worked penetration of microbiota improving agent | | | 249 | 239 | 101 | 391 | 291 |
| pH of solution having concentration of 1% by weight of | | | 6.6 | 6.9 | 7.0 | 7.0 | 6.9 |
| microbiota improving agent prepared by dilution with water | | | | | | | |
| Confirmation | Pathogenic | Porphyromonas gingivalis | 0.05 | 0.03 | 0.05 | 0.06 | 1.0 |
| test for | bacteria | Fusobacterium nucleatum | 0.05 | 0.04 | 0.05 | 0.05 | 1.0 |
| selective | | Prevotella intermedia | 0.06 | 0.03 | 0.06 | 0.06 | 1.0 |
| inhibition of | | Trichophyton rubrum | 0.05 | 0.02 | 0.05 | 0.07 | 1.0 |
| bacterial | | Trichophyton mentagrophytes | 0.06 | 0.03 | 0.06 | 0.06 | 1.0 |
| growth | | Staphylococcus aureus | 0.06 | 0.03 | 0.06 | 0.05 | 1.0 |
| | | Candida albicans | 0.05 | 0.05 | 0.08 | 0.05 | 1.0 |
| | | Candida glabrata | 0.05 | 0.05 | 0.09 | 0.05 | 0.9 |
| | Non- | Streptococcus salivarius | 1.1 | 1.1 | 1.1 | 1.0 | 1.0 |
| | pathogenic | Staphylococcus epidermidis | 1.0 | 1.0 | 1.0 | 1.0 | 1.0 |
| | bacteria | | | | | | |
| Inhibition assay of | | Porphyromonas gingivalis | 0.001 | 0.001 | 0.001 | 0.001 | 1.0 |
| protease production by | | Fusobacterium nucleatum | 0.002 | 0.001 | 0.002 | 0.002 | 1.0 |
| pathogenic bacteria | | Prevotella intermedia | 0.001 | 0.001 | 0.001 | 0.001 | 1.0 |
| Persistence | Pathogenic | Porphyromonas gingivalis | ∘∘ | ∘∘ | ∘∘ | ∘∘ | x |
| evaluation | bacteria | Fusobacterium nucleatum | ∘∘ | ∘∘ | ∘∘ | ∘∘ | x |
| | | Prevotella intermedia | ∘∘ | ∘∘ | ∘∘ | ∘∘ | x |
| | | Trichophyton rubrum | ∘∘ | ∘∘ | ∘∘ | ∘∘ | x |
| | | Trichophyton mentagrophytes | ∘∘ | ∘∘ | ∘∘ | ∘∘ | x |
| | | Staphylococcus aureus | ∘∘ | ∘∘ | ∘∘ | ∘∘ | x |
| | | Candida albicans | ∘∘ | ∘∘ | ∘∘ | ∘∘ | x |
| | | Candida glabrata | ∘∘ | ∘∘ | ∘∘ | ∘∘ | x |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |

| | | | | | |
|---|---|---|---|---|---|
| Spreadability evaluation | ∘∘ | ∘∘ | ∘ | ∘ | ∘ |

| | | | Comparative Example | | | | |
|---|---|---|---|---|---|---|---|
| | | | 2 | 3 | 4 | 5 | 6 |
| Substance (X) or (X′) | | X-1 | — | — | — | — | — |
| | | X-2 | — | — | — | — | — |
| | | X-3 | — | — | — | — | — |
| | | X-4 | — | — | — | — | — |
| | | X-5 | — | — | — | — | — |
| | | X-6 | — | — | — | — | — |
| | | X-7 | — | — | — | — | — |
| | | X-8 | — | — | — | — | — |
| | | X-9 | — | — | — | — | — |
| | | X-10 | — | — | — | — | — |
| | | X-11 | — | — | — | — | — |
| | | X-12 | — | — | — | — | — |
| | | X-13 | — | — | — | — | — |
| | | X-14 | — | — | — | — | — |
| | | X-15 | — | — | — | — | — |
| | | X-16 | — | — | — | — | — |
| | | X′-1 | — | — | — | — | — |
| | | X′-2 | — | — | — | — | — |
| Base (Y) or (Y′) | | Y-1 | — | — | — | — | — |
| | | Y-2 | 2.0 | — | — | — | — |
| | | Y-3 | — | 10.0 | — | — | — |
| | | Y-4 | — | — | 5.0 | 10.0 | — |
| | | Y-5 | — | — | — | — | 10.0 |
| | | Y-6 | — | — | — | — | — |
| | | Y-7-1 | — | — | — | — | — |
| | | Y-7-2 | — | — | — | — | — |
| | | Y-7-3 | — | — | — | — | — |
| | | Y′-1 | — | — | — | — | — |
| | | Y-7-4 | — | — | — | — | — |
| | | Y-8 | — | — | — | — | — |
| Volatile component | | Ion-exchanged water | 98.00 | 90.00 | 95.00 | 90.00 | 90.00 |
| Viscosity of microbiota improving agent (Pa · s) | | | 13.1 | 20 | 17.9 | 19.3 | 42 |
| Worked penetration of microbiota improving agent | | | 280 | 270 | 285 | 273 | 261 |
| pH of solution having concentration of 1% by weight of | | | 7.0 | 7.1 | 7.2 | 7.2 | 6.8 |
| microbiota improving agent prepared by dilution with water | | | | | | | |
| Confirmation | Pathogenic | Porphyromonas gingivalis | 0.9 | 0.9 | 0.9 | 0.9 | 0.9 |
| test for | bacteria | Fusobacterium nucleatum | 1.0 | 1.0 | 1.0 | 1.0 | 1.0 |
| selective | | Prevotella intermedia | 1.0 | 1.0 | 0.9 | 0.9 | 1.0 |
| inhibition of | | Trichophyton rubrum | 0.9 | 1.1 | 0.9 | 0.9 | 0.9 |
| bacterial | | Trichophyton mentagrophytes | 0.9 | 0.9 | 0.9 | 0.9 | 0.9 |
| growth | | Staphylococcus aureus | 1.1 | 0.9 | 0.9 | 0.9 | 0.9 |
| | | Candida albicans | 1.0 | 1.0 | 1.0 | 0.9 | 0.9 |
| | | Candida glabrata | 1.0 | 1.0 | 1.0 | 1.0 | 1.0 |
| | Non- | Streptococcus salivarius | 0.9 | 0.9 | 0.9 | 0.9 | 1.0 |
| | pathogenic | Staphylococcus epidermidis | 1.0 | 1.0 | 1.0 | 1.0 | 1.0 |
| | bacteria | | | | | | |
| Inhibition assay of | | Porphyromonas gingivalis | 1.0 | 1.1 | 1.0 | 1.0 | 1.0 |
| protease production by | | Fusobacterium nucleatum | 1.0 | 1.0 | 1.0 | 1.0 | 1.0 |
| pathogenic bacteria | | Prevotella intermedia | 0.9 | 1.0 | 1.0 | 1.0 | 1.0 |
| Persistence | Pathogenic | Porphyromonas gingivalis | x | x | x | x | x |
| evaluation | bacteria | Fusobacterium nucleatum | x | x | x | x | x |
| | | Prevotella intermedia | x | x | x | x | x |
| | | Trichophyton rubrum | x | x | x | x | x |
| | | Trichophyton mentagrophytes | x | x | x | x | x |
| | | Staphylococcus aureus | x | x | x | x | x |
| | | Candida albicans | x | x | x | x | x |
| | | Candida glabrata | x | x | x | x | x |
| | Non- | Streptococcus salivarius | ∘∘ | ∘∘ | ∘∘ | ∘∘ | ∘∘ |
| | pathogenic | Staphylococcus epidermidis | ∘∘ | ∘∘ | ∘∘ | ∘∘ | ∘∘ |
| | bacteria | | | | | | |

| | | | | | |
|---|---|---|---|---|---|
| Spreadability evaluation | ∘ | ∘ | ∘ | ∘ | ∘ |

TABLE 1-6

| | | | Comparative Example | | | | | | | | | |
|---|---|---|---|---|---|---|---|---|---|---|---|---|
| | | | 7 | 8 | 9 | 10 | 11 | 12 | 13 | 14 | 15 | 16 |
| Substance (X) or (X′) | | X-1 | — | — | — | — | 3.0 | 3.0 | 3.0 | 3.0 | — | — |
| | | X-2 | — | — | — | — | — | — | — | — | — | — |
| | | X-3 | — | — | — | — | — | — | — | — | — | — |
| | | X-4 | — | — | — | — | — | — | — | — | — | — |
| | | X-5 | — | — | — | — | — | — | — | — | — | — |
| | | X-6 | — | — | — | — | — | — | — | — | — | — |
| | | X-7 | — | — | — | — | — | — | — | — | — | — |
| | | X-8 | — | — | — | — | — | — | — | — | — | — |
| | | X-9 | — | — | — | — | — | — | — | — | — | — |
| | | X-10 | — | — | — | — | — | — | — | — | — | — |
| | | X-11 | — | — | — | — | — | — | — | — | — | — |
| | | X-12 | — | — | — | — | — | — | — | — | — | — |
| | | X-13 | — | — | — | — | — | — | — | — | — | — |
| | | X-14 | — | — | — | — | — | — | — | — | — | — |
| | | X-15 | — | — | — | — | — | — | — | — | — | — |
| | | X-16 | — | — | — | — | — | — | — | — | — | — |
| | | X′-1 | — | — | — | — | — | — | — | — | 3.0 | — |
| | | X′-2 | — | — | — | — | — | — | — | — | — | 3.0 |
| Base (Y) or (Y′) | | Y-1 | — | — | — | — | — | — | — | — | — | — |
| | | Y-2 | — | — | — | — | — | — | — | — | — | — |
| | | Y-3 | — | — | — | — | — | 1.0 | — | — | 10.0 | 10.0 |
| | | Y-4 | — | — | — | — | — | — | — | — | — | — |
| | | Y-5 | — | — | — | — | — | — | — | — | — | — |
| | | Y-6 | 50.0 | — | — | — | — | — | — | — | — | — |
| | | Y-7-1 | — | 100.0 | — | — | — | — | — | — | — | — |
| | | Y-7-2 | — | — | 100.0 | — | — | — | — | — | — | — |
| | | Y-7-3 | — | — | — | 100.0 | — | — | — | — | — | — |
| | | Y′-1 | — | — | — | — | 97.0 | — | — | — | — | — |
| | | Y-7-4 | — | — | — | — | — | — | 97.0 | — | — | — |
| | | Y-8 | — | — | — | — | — | — | — | — | — | — |
| Volatile component | | Ion-exchanged water | 50.00 | — | — | — | — | 96.00 | — | 97.00 | 87.00 | 87.00 |
| Viscosity of microbiota improving agent (Pa · s) | | | 2.2 | 3500 | 90 | 8.9 | 100 | 1.1 | 4500 | Less | 20 | 18 |
| | | | | | | | | | | than 1 | | |
| Worked penetration of microbiota improving agent | | | 298 | 282 | 257 | 250 | 250 | 450 | 95 | — | 271 | 265 |
| pH of solution having concentration of 1% by weight of | | | 7.1 | 7.0 | 6.8 | 6.5 | 6.8 | 7.0 | 6.9 | 7.0 | 6.9 | 7.3 |
| microbiota improving agent prepared by dilution with water | | | | | | | | | | | | |
| Confirmation | Pathogenic | Porphyromonas gingivalis | 1.0 | 0.9 | 1.0 | 0.9 | 0.1 | 0.1 | 1.0 | 0.1 | 0.03 | 0.04 |
| test for | bacteria | Fusobacterium nucleatum | 1.0 | 0.9 | 1.0 | 1.0 | 0.1 | 0.1 | 1.1 | 0.1 | 0.04 | 0.05 |
| selective | | Prevotella intermedia | 1.0 | 1.0 | 1.0 | 1.1 | 0.1 | 0.1 | 1.0 | 0.1 | 0.03 | 0.03 |
| inhibition of | | Trichophyton rubrum | 0.9 | 1.0 | 0.9 | 0.9 | 0.1 | 0.08 | 0.9 | 0.09 | 0.05 | 0.05 |
| bacterial | | Trichophyton mentagrophytes | 1.0 | 0.9 | 0.9 | 1.0 | 0.1 | 0.09 | 0.8 | 0.11 | 0.03 | 0.03 |
| growth | | Staphylococcus aureus | 1.0 | 0.9 | 0.9 | 1.1 | 0.1 | 0.1 | 1.0 | 0.09 | 0.03 | 0.03 |
| | | Candida albicans | 1.0 | 1.0 | 0.9 | 0.9 | 0.1 | 0.1 | 0.9 | 0.1 | 0.04 | 0.05 |
| | | Candida glabrata | 0.9 | 1.0 | 1.0 | 1.0 | 0.1 | 0.09 | 1.0 | 0.1 | 0.04 | 0.05 |
| | Non- | Streptococcus salivarius | 0.9 | 0.9 | 0.9 | 1.0 | 1.1 | 0.1 | 1.0 | 0.1 | 0.02 | 0.03 |
| | pathogenic | Staphylococcus epidermidis | 1.0 | 1.0 | 1.0 | 1.0 | 0.9 | 0.1 | 0.9 | 0.1 | 0.03 | 0.05 |
| | bacteria | | | | | | | | | | | |
| Inhibition assay of | | Porphyromonas gingivalis | 1.0 | 1.1 | 1.0 | 1.0 | 0.001 | 0.001 | 1.0 | 0.002 | 0.001 | 0.002 |
| protease production by | | Fusobacterium nucleatum | 1.0 | 1.0 | 1.0 | 0.9 | 0.001 | 0.001 | 0.9 | 0.001 | 0.001 | 0.001 |
| pathogenic bacteria | | Prevotella intermedia | 1.0 | 1.1 | 1.0 | 1.0 | 0.001 | 0.001 | 1.0 | 0.001 | 0.001 | 0.001 |
| Persistence | Pathogenic | Porphyromonas gingivalis | x | x | x | x | x | x | x | x | ∘ | ∘ |
| evaluation | bacteria | Fusobacterium nucleatum | x | x | x | x | x | x | x | x | ∘ | ∘ |
| | | Prevotella intermedia | x | x | x | x | x | x | x | x | ∘ | ∘ |
| | | Trichophyton rubrum | x | x | x | x | x | x | x | x | ∘ | ∘ |
| | | Trichophyton mentagrophytes | x | x | x | x | x | x | x | x | ∘ | ∘ |
| | | Staphylococcus aureus | x | x | x | x | x | x | x | x | ∘ | ∘ |
| | | Candida albicans | x | x | x | x | x | x | x | x | ∘ | ∘ |
| | | Candida glabrata | x | x | x | x | x | x | x | x | ∘ | ∘ |
| | Non- | Streptococcus salivarius | ∘∘ | ∘∘ | ∘∘ | ∘∘ | ∘∘ | x | ∘∘ | x | x | x |
| | pathogenic | Staphylococcus epidermidis | ∘∘ | ∘∘ | ∘∘ | ∘∘ | ∘∘ | x | ∘∘ | x | x | x |
| | bacteria | | | | | | | | | | | |

| | | | | | | | | | | |
|---|---|---|---|---|---|---|---|---|---|---|
| Spreadability evaluation | ∘ | ∘∘ | ∘ | ∘ | x | ∘ | Δ | x | ∘ | ∘ |

TABLE 2

| | | | Weight | Viscosity (mPa · s) |
|---|---|---|---|---|
| | | | average | of solution having |
| | | HLB value | molecular | concentration of 1% by |
| | | of non- | weight of | weight of non-volatile |
| | | volatile | non-volatile | component prepared by |
| | | component | component | dilution with water |
| Base | Y-1 | 27.1 | 49,000 | 200 |
| (Y) or | Y-2 | 44.0 | 200,000 | 1,000 |
| (Y′) | Y-3 | 58.2 | 100,000 | 500 |
| | Y-4 | 34.6 | 2,000,000 | 1,500 |
| | Y-5 | 30.0 | 1,000,000 | 800 |
| | Y-6 | 18.9 | 20,000 | 1,000 |
| | Y-7-1 | 7.8 | 28,000 | 10 |
| | Y-7-2 | 7.8 | 28,000 | 10 |
| | Y-7-3 | 7.8 | 28,000 | 10 |
| | Y′-1 | 0 | 300 | 0.8 |
| | Y-7-4 | 7.8 | 28,000 | 10.0 |
| | Y-8 | 34.3 | 140,000 | 1,000 |

TABLE 3

| | | Amount of magnesium (g) captured |
|---|---|---|
| | | per 100 g of substance (X) |
| Substance (X) | X-1 | 5 |
| | X-2 | 3 |
| | X-3 | 3 |
| | X-4 | 2 |
| | X-5 | 2 |
| | X-6 | 2 |
| | X-7 | 2 |
| | X-8 | 15 |
| | X-9 | 20 |
| | X-10 | 30 |
| | X-11 | 15 |
| | X-12 | 10 |
| | X-13 | 8 |
| | X-14 | 2 |
| | X-15 | 9 |
| | X-16 | 5 |

As shown in Tables 1-1 to 1-6, in Examples 1 to 44 containing the substances (X) and (Y), the growth of pathogenic bacteria was inhibited selectively, whereas the growth of non-pathogenic bacteria was not inhibited, thus achieving an ideal microbiota balance, compared with Comparative Examples 1 to 16. Therefore, the microbiota improvement effect was confirmed in Examples 1 to 44.

Furthermore, in Comparative Examples 15 and 16 in which a common antibacterial agent (benzalkonium chloride or cetylpyridinium chloride) was used, the growth of not only pathogenic bacteria but also non-pathogenic bacteria were inhibited. On the other hand, in Examples 1 to 44, the growth of non-pathogenic bacteria was maintained, suggesting the effect of selectively inhibiting pathogenic bacteria.

Furthermore, in Examples 1 to 44, an inhibitory effect on the production of toxins (proteases) by pathogenic bacteria was confirmed. Also, the long-term persistence of the microbiota improving effect was confirmed compared with Comparative Examples 11, 12, and 14 in which a protease production inhibitory effect is exhibited.

Therefore, the microbiota improving agents of the present invention were found to have the effect of selectively inhibiting the growth of pathogenic bacteria and inhibiting toxin production by pathogenic bacteria without affecting non-pathogenic bacteria and to allow the effect to persist for a long period of time. Once the microbiota improving agent of the present invention is applied to the affected area, it can exhibit a microbiota balance improving effect for a long period of time. Thus, the microbiota improving agents of the present invention are particularly useful as a microbiota improving agent.

1. A microbiota improving agent for improving at least one of mucosal microbiota, skin microbiota, or nail microbiota, the microbiota improving agent comprising:
a substance (X); and
a base (Y) containing a non-volatile component,
the substance (X) including at least one selected from the group consisting of a compound (X1) having two or more hydroxy groups contained in at least one oxoacid group and a salt of the compound (X1),
a compound (X2) having one hydroxy group contained in an oxoacid group and a salt of the compound (X2),
a heterocyclic compound (X3) having no oxoacid group and a salt of the compound (X3), and
a ketone (X4) having no oxoacid group,
wherein in the compound (X1), a total mass percentage of the at least one oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 45% by mass or more based on a molecular weight of the compound (X1),
the compound (X2) has an atom having an unshared electron pair, the atom excluding atoms contained in the oxoacid group and atoms contained in an amino group not constituting an imidazole skeleton and in an amino group not constituting a pyridine skeleton,
in the compound (X2), a total mass percentage of the oxoacid group, a hydroxy group not contained in the oxoacid group, a carbonyl group not contained in the oxoacid group, carbon atoms contained in an imidazole skeleton and a pyridine skeleton, and nitrogen atoms contained in the imidazole skeleton and the pyridine skeleton is 55% by mass or more based on a molecular weight of the compound (X2),
the heterocyclic compound (X3) has an imidazole skeleton or a pyridine skeleton and at least one of a hydroxy group or a carbonyl group, or has two or more skeletons selected from the group consisting of an imidazole skeleton and a pyridine skeleton,
the ketone (X4) has a linear β-diketone structure,
the substance (X) has a molecular weight of 550 or less,
the non-volatile component has an HLB value of 7 or more, and
the microbiota improving agent satisfies the following conditions (i) and (ii):
(i) the microbiota improving agent has a worked penetration of 100 to 400 as measured in accordance with JIS K 2220; and
(ii) the microbiota improving agent has a viscosity at 37° C. of 1 to 4,000 Pa·s as measured with a cone-and-plate rheometer.

2. The microbiota improving agent according to claim 1,
wherein a solution having a concentration of 1% by weight of the microbiota improving agent prepared by diluting the microbiota improving agent with water has a pH of 6 to 8.

3. The microbiota improving agent according to claim 1,
wherein a weight percentage of the substance (X) is 0.01 to 5% by weight based on a weight of the microbiota improving agent.
